(19) United States
(12) Patent Application Publication
(10) Pub. No.: US 2004/0110787 A1
(43) Pub. Date: Jun. 10, 2004

(54) Heteroaryl-ethanolamine derivatives as antiviral agents

(76) Inventors: Mark E. Schnute, O'Fallon, MO (US); Michele M. Cudahy, Plainwell, MI (US); David John Anderson, Kalamazoo, MI (US); Thomas M. Judge, Otsego, MI (US); Thomas J. Fleck, Scotts, MI (US); Paul Matthew Herrinton, Portage, MI (US); Sajiv K. Nair, Vista, CA (US); Allen Scott, Dover, TN (US); William R. Perrault, Kalamazoo, MI (US); Steve P. Tanis, Carlsbad, CA (US); James A. Nieman, Carlsbad, CA (US); Sarah A. Collier, Decatur, GA (US); Bruce F. Fleck, Scotts, MI (US)

Correspondence Address: PHARMACIA & UPJOHN, 301 HENRIETTA ST, 0228-32-LAW, KALAMAZOO, MI, 49007, US

(21) Appl. No.: 10/649,301

(22) Filed: Aug. 27, 2003

Related U.S. Application Data
(60) Provisional application No. 60/408,065, filed on Sep. 4, 2002.

(51) Int. Cl. A61K 31/496; A61K 31/4743; C07D 498/02

(52) U.S. Cl. 514/301; 546/114; 514/253.04; 544/362

(57) ABSTRACT

The present invention provides a compound of formula as described herein, which are useful as antiviral agents, in particular, as agents against viruses of the herpes family.

### CROSS REFERENCE

This application claims the benefit of the following provisional application: U.S. Serial No. 60/408,065, filed Sep. 4, 2002 under 35 USC 119(e)(i), which is incorporated herein by reference in its entirety.

### FIELD OF THE INVENTION

The present invention discloses six-(6) membered heteroaryl-ethanolamine derivatives, and more specifically, provides compounds of formula (I) described herein below. These compounds are useful as antiviral agents, in particular, as agents against viruses of the herpes family.

### BACKGROUND OF THE INVENTION

The herpesviruses comprise a large family of double stranded DNA viruses. They are also a source of the most common viral illnesses in man. Eight of the herpes viruses, herpes simplex virus types 1 and 2 (HSV-1 and HSV-2), varicella zoster virus (VZV), human cytomegalovirus (HCMV), Epstein-Barr virus (EBV), and human herpes viruses 6, 7, and 8 (HHV-6, HHV-7, and HHV-8), have been shown to infect humans.

HSV-1 and HSV-2 cause herpetic lesions on the lips and genitals, respectively. They also occasionally cause infections of the eye and encephalitis. HCMV causes birth defects in infants and a variety of diseases in immunocompromised patients such as retinitis, pneumonia, and gastrointestinal disease. VZV is the causative agent of chicken pox and shingles. EBV causes infectious mononucleosis. It can also cause lymphomas in immunocompromised patients and has been associated with Burkitt's lymphoma, nasopharyngeal carcinoma, and Hodgkins disease. HHV-6 is the causative agent of roseola and may be associated with multiple sclerosis and chronic fatigue syndrome. HHV-7 disease association is unclear, but it may be involved in some cases of roseola. HHV-8 has been associated with Karposi's sarcoma, body cavity based lymphomas, and multiple myeloma.

Infection by or reactivation of herpesviruses is associated with several cardiovascular diseases or conditions in the host such as atherosclerosis and restenosis resulting in inflammation of coronary vessel walls. It is thought that in many patients suffering from restenosis following coronary atherectomy viral infection particularly by CMV plays an important role in the proliferation of the disease. Atherosclerosis is believed to be associated with the overall infectious disease burden in the host and particularly by the herpesviruses such as HSV, CMV, and EBV.

Infection in the animal population (livestock and companion) by strains of herpesviruses is endemic including cattle (Bovine herspesvirus 1-5, BHV), sheep (Ovine herpesvirus 1 and 2), dog (Canine herpesvirus 1), horse (Equine herpesvirus 1-8, EHV), cat (Feline herpesvirus 1, FHV), swine (pseudorabies virus, PRV), and many species of fowl. In the case of bovine herpesvirus infection, animals may suffer from ocular, respiratory, or digestive disorders. Pseudorabies is an extremely contagious viral pathogen infecting several species such as cattle, horses, dogs, cats, sheep, and goats leading to rapid death. The virus is benign in adult swine, however, it remains contagious and leads to high mortality in pigs under three weeks. Infection of horses by equine herpesvirus may lead to neurological syndromes, respiratory disease, and neonatal disease. Herpesvirus infection in cats leads to the disease known as feline viral rhinotracheitis (FVR) which is characterized by rhinitis, tracheitis, laryngitis, and conjunctivitis.

Due to the unique position of the six- (6) membered heteroaryl substitutent on the formula I described herein below, compounds of the present invention demonstrate unexpected activity against the above reference herpesviral infections, particularly, human cytomegaloviral infection.

### INFORMATION DISCLOSURE

U.S. Pat. No. 6,239,142 disclosed compounds and their use to treat herpesvirus infections.

WO02/06513 disclosed method of screening 4-hydroxyquinline, 4-oxo-dihydroquinoline, and 4-oxo-dihydrothienopyridine derivatives as non-nucleoside herpesvirus DNA polymerase inhibitors.

Tetrahedron Lett. 1983, 24, 3233-3236 describes conditions to transform tertiary N-benzylamines into benzylchlorides.

WO95/28405 disclosed bicyclic thiophene derivatives and use as gonadotropin releasing hormone antagonists.

EP 443568 disclosed fused thiophene derivatives, their production and use.

WO02/04445 disclosed a variety of tricyclic core structures which have antiviral activity against herpesviruses.

WO02/04444, WO02/04443, and WO02/04422 disclosed a variety of bicyclic core structures which have antiviral activity against herpesviruses.

U.S. Pat. No. 6,248,739 disclosed compounds in which the core structure is a quinoline and useful as antivirals against herpesviruses.

WO00/53178, WO00/53179, WO00/53180, WO00/53181, WO00/53185, and WO00/53602 disclosed 6-azaindole compounds as antagonists of gonadotropin releasing hormone.

U.S. Pat. No. 6,346,534 and WOOO/69859 disclosed imidazo- and pyrrolo[1,2-a]pyrimid-4-ones as gonadotropin-releasing hormone receptor antagonists.

WO 94/12461 disclosed a variety of bicyclic core structures useful as potential treatments of AIDS, asthma, arthritis, and other inflammatory diseases.

### SUMMARY OF THE INVENTION

The present invention provides a compound of formula I

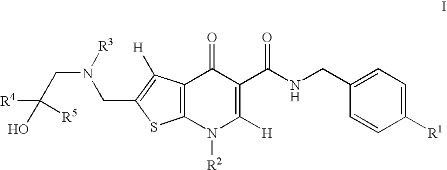

its enantiomeric, diasteromeric or tautomeric isomer, or a pharmaceutically acceptable salt thereof wherein,

R¹ is

(a) Cl,

(b) Br,

(c) F, or

(d) CN;

R² is

(a) C₁₋₄alkyl optionally substituted by one or more OH or C₁₋₄alkoxy, or

(b) (CH₂)ₘOCH₂CH₂OH;

R³ is C₁₋₂alkyl;

R⁴ is a six- (6) membered heteroaryl bonded via a carbon atom having 1, 2, or 3 nitrogen atoms, wherein R⁴ is optionally fused to a benzene ring, and optionally substituted with one or more R⁶;

R⁵ is

(a) H, or

(b) C₁₋₂alkyl optionally substituted by OH;

R⁶ is

(a) halo,

(b) OCF₃,

(c) cyano,

(d) nitro,

(e) CONR⁷R⁸,

(D NR⁷R⁸,

(g) C₁₋₇alkyl, which is optionally partially unsaturated and is optionally substituted by one or more R⁹,

(h) O(CH₂CH₂O)ₙR¹⁰,

(i) OR¹⁰, or

(j) CO₂R¹⁰,

R⁷ and R⁸ are independently

(a) H,

(b) phenyl optionally substituted by halo, C₁₋₇alkyl, or C₁₋₇alkoxy,

(c) C₁₋₇alkyl which is optionally substituted by one or more OR¹⁰, phenyl, or halo substituents,

(d) C₃₋₈cycloalkyl,

(e) (C═O)R¹¹, or

(f) R⁷ and R⁸ together with the nitrogen to which they are attached form a het, wherein het is a five- (5), or six- (6) membered heterocyclic ring having 1, 2, or 3 heteroatoms selected from the group consisting of oxygen, sulfur, or nitrogen, wherein het is optionally substituted with C₁₋₄ alkyl;

R⁹ is

(a) oxo,

(b) phenyl optionally substituted by halo, C₁₋₇alkyl, or C₁₋₇alkoxy,

(c) OR¹⁰,

(d) O(CH₂CH₂)OR¹⁰,

(e) SR¹⁰,

(f) NR₇R₈,

(g) halo,

(h) CO₂R₁₀,

(i) CONR¹⁰R¹⁰, or

(j) C₃₋₈cycloalkyl optionally substituted by OR¹⁰;

R¹⁰ is

(a) H,

(b) C₁₋₇alkyl,

(c) C₃₋₈cycloalkyl, or

(d) phenyl optionally substituted by halo, C₁₋₇alkyl, or C₁₋₇alkoxy,

R¹¹ is

(a) C₁₋₇alkyl;

(b) C₃₋₈cycloalkyl, or

(c) phenyl optionally substituted by halo, C₁₋₇alkyl, or C₁₋₇alkoxy,

n is 1, 2, 3, 4 or 5; and

m is 1 or 2.

In another aspect, the present invention also provides:

A pharmaceutical composition which comprises a pharmaceutically acceptable carrier and an effective amount of a compound of formula I,

a method of treating and preventing herpesviral infections in a mammal comprising administering to a mammal in need thereof a compound of formula I, or a pharmaceutically acceptable salt thereof,

a method for inhibiting a viral DNA polymerase comprising contacting, in vivo or in vitro, the polymerase with an effective inhibitory amount of a compound of formula I, or a pharmaceutically acceptable salt thereof,

a compound of formula I or a pharmaceutically acceptable salt thereof for use in medical treatment or prevention of a herpesviral infection in a mammal.

The invention also provides novel intermediates and processes disclosed herein that are useful for preparing compounds of formula I.

### DETAILED DESCRIPTION OF THE INVENTION

For the purpose of the present invention, the carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₇)alkyl refers to alkyl of one to seven carbon atoms, inclusive, or methyl, ethyl, propyl, butyl, pentyl, hexyl, and heptyl, straight and branched forms thereof.

The term “halo” or “halogen” refers to the elements fluorine (F), chlorine (Cl), bromine (Br) and iodine (I).

The term “C₃₋₈cycloalkyl” refers to a non-aromatic carbocyclic ring having from 3 to 8 carbon atoms.

The term “alkoxy” refers to the group RO—, wherein R is alkyl or cycloalkyl as defined above.

The term “heteroaryl” refers to aromatic heterocyclic groups.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The compounds of the present invention are generally named according to the IUPAC or CAS nomenclature system.

“Pharmaceutically acceptable salts” refers to those salts which possess the biological effectiveness and properties of the parent compound and which are not biologically or otherwise undesirable.

“Mammal” refers to human and animals. Animals specifically refer to, for example, food animals or companion animals.

“Optionally” or “may be” means that the subsequently described event or circumstance may, but need not, occur, and that the description includes instances where the event or circumstance occurs and instances in which it does not.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

Specifically, formula I of the present invention has a stereogenic center as shown in formula IA:

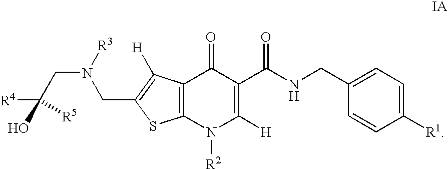

Specifically, a composition comprising over 51% of a compound of formula IA.

Specifically, a composition comprising over 75% of a compound of formula IA.

Specifically, a composition comprising over 90% of a compound of formula IA.

Specifically, a composition comprising over 98% of a compound of formula IA.

Specifically, R¹ is chloro.

Specifically, R² is C₁₋₃alkyl.

Specifically, R² is methyl, ethyl, or n-propyl.

Specifically, R² is methyl.

Specifically, R² is C₁₋₃alkyl substituted with one or two hydroxy.

Specifically, R² is 2-hydroxyethyl, 3-hydroxypropyl, or 2,3-dihydroxypropyl.

Specifically, R² is C₁₋₄alkyl substituted by C₁₋₄alkoxy.

Specifically, R² is C₁₋₄alkyl substituted by methoxy.

Specifically, R² is 2-methoxyethyl.

Specifically, R² is CH₂CH₂OCH₂CH₂OH.

Specifically, R³ is methyl.

Specifically, R³ is ethyl.

Specifically, R⁴ is a six- (6) membered heteroaryl bonded via a carbon atom having one (1), two (2), or three (3) nitrogen atoms.

Specifically, R⁴ is pyridin-2-yl, pyridin-3-yl, pyridin-4-yl, pyrazin-2-yl, pyrimidin-2-yl, pyrimidin-4-yl, 2-pyridazin-3-yl, pyrimidin-5-yl, pyridazin-4-yl, (1,2,4-triazin-6-yl), (1,2,4-triazin-3-yl), (1,3,5-triazin-2-yl), or (1,2,4-triazin-5-yl).

Specifically, R⁴ is a six- (6) membered heteroaryl bonded via a carbon atom having one (1) or two (2) nitrogen atoms.

Specifically, R⁴ is pyridin-2-yl, pyridin-3-yl, pyridin-4-yl, pyrazin-2-yl, pyrimidin-2-yl, pyrimidin-4-yl, 2-pyridazin-3-yl, pyrimidin-5-yl, or pyridazin-4-yl.

Specifically, R⁴ is pyridin-2-yl.

Specifically, R⁴ is a six- (6) membered heteroaryl bonded via a carbon atom having one (1), two (2), or three (3) nitrogen atoms wherein R⁴ is fused to a benzene ring.

Specifically, R⁴ is isoquinolin-3-yl, quinolin-3-yl, quinolin-2-yl, quinazolin-2-yl, quinoxalin-2-yl, cinnolin-3-yl, (1,2,4-benzotriazin-3-yl), isoquinolin-1-yl, isoquinolin-4-yl, quinolin-4-yl, quinazolin-4-yl, phthalazin-1-yl, or cinnolin-4-yl.

Specifically, R⁴ is a six- (6) membered heteroaryl bonded via a carbon atom having one (1) or two (2) nitrogen atoms wherein R⁴ is fused to a benzene ring.

Specifically, R⁴ is isoquinolin-3-yl, quinolin-3-yl, quinolin-2-yl, quinazolin-2-yl, quinoxalin-2-yl, cinnolin-3-yl, isoquinolin-1-yl, isoquinolin-4-yl, quinolin-4-yl, quinazolin-4-yl, phthalazin-1-yl, or cinnolin-4-yl.

Specifically, R⁴ is a six- (6) membered heteroaryl bonded via a carbon atom having one (1) nitrogen atom wherein R⁴ is fused to a benzene ring.

Specifically, R⁴ is isoquinolin-3-yl, quinolin-3-yl, quinolin-2-yl, isoquinolin-1-yl, isoquinolin-4-yl, quinolin-4-yl.

Specifically, R⁴ is optionally substituted with R⁶.

Specifically, R⁴ is pyridin-3-yl, pyridin-4-yl, pyridin-2-yl, 6-methylpyridin-2-yl, pyrimidin-2-yl, pyrazin-2-yl, or quinolin-2-yl.

Specifically, R⁵ is hydrogen.

Specifically, R⁵ is methyl or ethyl.

Specifically, R⁵ is hydroxymethyl, 1-hydroxyethyl, or 2-hydroxyethyl.

Specifically, R⁶ is OH, halo, C₁₋₄alkyl, C₁₋₄alkoxy, cyano, nitro, OCF₃, NR⁷R⁸, or CONR⁷R⁸.

Specifically, R⁶ is methyl.

Specifically, R⁶ is amino, morpholine, piperidine, piperazine, or pyrrolidine.

Examples of the present invention include, but are not limited to the following:

(1) rac-N-(4-chlorobenzyl)-2-(((2-hydroxy-2-pyridin-3-ylethyl)(methyl) amino)-methyl)-7-methyl-4-oxo-4,7-dihydrothieno[2,3-b]pyridine-5-carboxamide,

(2) (+)-N-(4-chlorobenzyl)-2-(((2-hydroxy-2-pyridin-3-ylethyl)(methyl)amino)-methyl)-7-methyl-4-oxo-4,7-dihydrothieno[2,3-b]pyridine-5-carboxamide,

(3) rac-N-(4-chlorobenzyl)-2-(((2-hydroxy-2-pyridin-4-ylethyl)(methyl)amino)-methyl)-7-methyl-4-oxo-4,7-dihydrothieno[2,3-b]pyridine-5-carboxamide,

(4) rac-N-(4-chlorobenzyl)-2-(((2-hydroxy-2-pyridin-2-ylethyl)(methyl)amino)-methyl)-7-methyl-4-oxo-4,7-dihydrothieno[2,3-b]pyridine-5-carboxamide,

(5) (+)-N-(4-chlorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide,

(6) rac-N-(4-chlorobenzyl)-2-(((2-hydroxy-2-(6-methylpyridin-2-yl)ethyl)(methyl)amino)methyl)-7-methyl-4-oxo-4,7-dihydrothieno[2,3-b]pyridine-5-carboxamide,

(7) rac-N-(4-chlorobenzyl)-2-(((2-hydroxy-2-quinolin-2-ylethyl)(methyl)amino)methyl)-7-methyl-4-oxo-4,7-dihydrothieno[2,3-b]pyridine-5-carboxamide,

(8) rac-N-(4-chlorobenzyl)-2-(((2-hydroxy-2-pyrimidin-2-ylethyl)(methyl)amino)methyl)-7-methyl-4-oxo-4,7-dihydrothieno[2,3-b]pyridine-5-carboxamide,

(9) N′-(4-chlorobenzyl)-2-((((2R)-2-hydroxy-2-pyrimidin-2-ylethyl)(methyl)amino)methyl)-7-methyl-4-oxo-4,7-dydrothieno[2,3-b]pyridine-5-carboxamide,

(10) rac-N-(4-chlorobenzyl)-2-(((2-hydroxy-2-pyrazin-2-ylethyl)(methyl)amino)methyl)-7-methyl-4-oxo-4,7-dihydrothieno[2,3-b]pyridine-5-carboxamide,

(11) N-(4-Chlorobenzyl)-2-((((2R)-2-hydroxy-2-pyrazin-2N-ylethyl)N(methyl)amino)methyl)-7-methyl-4-oxo-4,7-dihydrothieno[2,3-b]pyridine-5-carboxamide,

(12) N-(4-chlorobenzyl)-2-(((2-hydroxy-2-pyridazin-3-ylethyl)(methyl)amino)methyl)-7-methyl-4-oxo-4,7-dihydrothieno[2,3-b]pyridine-5-carboxamide,

(13) rac-N-(4-chlorobenzyl)-7-ethyl-2-(((2-hydroxy-2-pyrazin-2-ylethyl)(methyl)amino)methyl)-4-oxo-4,7-dihydrothieno[2,3-b]pyridine-5-carboxamide,

(14) rac-N-(4-chlorobenzyl)-7-ethyl-2-(((2-hydroxy-2-pyridin-2-)ylethyl)(methyl)amino)methyl)-4-oxo-4,7-dihydrothieno[2,3-b]pyridine-5-carboxamide,

(15) rac-N-(4-chlorobenzyl)-7-propyl-2-(((2-hydroxy-2-pyrridin-2-ylethyl)(methyl)amino)methyl)-4-oxo-4,7-dihydrothieno[2,3-b]pyridine-5-carboxamide,

(16) rac-N-(4-chlorobenzyl)-2-(((2-hydroxy-2-pyrazin-2-ylethyl)(methyl)amino)methyl)-4-oxo-7-propyl-4,7-dihydrothieno[2,3-b]pyridine-5-carboxamide,

(17) N-(4-chlorobenzyl)-7-(2,3-dihydroxypropyl)-2-((((2R)-2-hydroxy-2-pyridin-2-ylethyl)(methyl)amino)methyl)-4-oxo-4,7-dihydrothieno[2,3-b]pyridine-5-carboxamide,

(18) N-(4-chlorobenzyl)-7-(3-hydroxypropyl)-2-((((2R)-2-hydroxy-2-pyridin-2-ylethyl)(methyl)amino)methyl)-4-oxo-4,7-dihydrothieno[2,3-b]pyridine-5-carboxamide,

(19) rac-N-(4-chlorobenzyl)-7-(3-hydroxypropyl)-2-(((2-hydroxy-2-pyridin-2-ylethyl)(methyl)amino)methyl)-4-oxo-4,7-dihydrothieno[2,3-b]pyridine-5-carboxamide.

(20) N-(4-chlorobenzyl)-7-(2-hydroxyethyl)-2-((((2R)-2-hydroxy-2-pyridin-2-ylethyl)(methyl)amino)methyl)-4-oxo-4,7-dihydrothieno[2,3-b]pyridine-5-carboxamide,

(21) rac-N-(4-chlorobenzyl)-2-(((2-hydroxy-2-pyrazin-2-ylethyl)(methyl)amino)methyl)-7-(2-methoxyethyl)-4-oxo-4,7-dihydrothieno[2,3-b]pyridine-5-carboxamide,

(22) N-(4-Chlorobenzyl)-2-((((2R)-2-hydroxy-2-pyrazin-2-ylethyl)(methyl)amino)methyl)-4-oxo-7-(2-(2-(tetrahydro-2H-pyran-2-yloxy)ethoxy)ethyl)-4,7-dihydiothieno[2,3-b]pyridine-5-carboxamide,

(23) N-(4-fluorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide,

(24) N-(4-cyanobenzyl)-2-((((4)R)-2-hydroxy-2-pyridin-2-ylethyl)(methyl)amino)methyl)-7-methyl-4-oxo-4,7-dihydrothieno[2,3-b]pyridine-5-carboxamide,

(25) N-(4-bromobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide, and their pharmaceutically acceptable salts thereof.

Charts A-O describe the preparation of the compounds of Formula (I) of the present invention. All of the starting materials are prepared by procedures described in these charts, by procedures well known to one of ordinary skill in organic chemistry or can be obtained commercially. All of the final compounds of the present invention are prepared by procedures described in these charts or by procedures analogous thereto, which would be well known to one of ordinary skill in organic chemistry.

Compounds of Formula (I) are prepared as described in Chart A. Compounds of the formula A.1 in which X is a leaving group (e.g. mesylate, chloride, or bromide) are treated with a secondary amine of the formula R⁴R⁵C(OH)CH₂NH(R³) in the presence of a non-nucleophilic base (e.g. diisopropylethylamine) in a polar solvent (e.g. DMF) to afford products of the formula A.2. It would be understood by those skilled in the art that in some cases transient protection of hydroxyl and other Lewis basic or acidic functionality present in R⁴R⁵C(OH)CH₂NH(R³) may be required to facilitate the coupling described in Chart A for which procedures are well established (Greene, T. W.; Wuts, P. G. M. Protective Groups in Organic Synthesis, 1999).

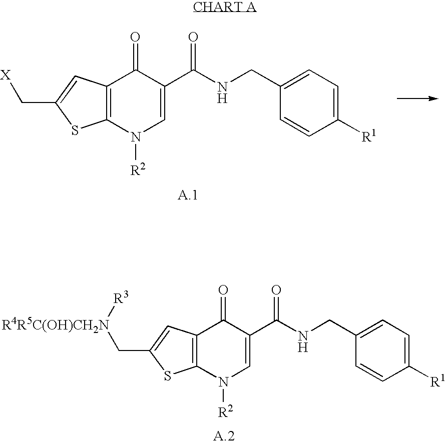

Alternatively, compounds of Formula (I) are prepared as described in Chart B. Compounds of the formula A.1 in which X is a leaving group (e.g. mesylate, chloride, or bromide) are treated with a primary amine of the formula R⁴R⁵C(OH)CH₂NH₂ in the presence of a non-nucleophilic base (e.g. diisopropylethylamine) in a polar solvent (e.g. DMF) to afford products of the formula B. 1. The resulting secondary amine is then alkylated by reactions generally known by those skilled in the art such as (1) the reaction of B.1 with a corresponding alkylhalide, dialkylsulfonate, or alkylarylsulfonate or (2) the reaction of B.1 with an aldehyde (e.g. formaldehyde or acetaldehyde) in the presence of a reducing agent (e.g. sodium cyanoborohydride or sodium triacetoxyborohydride) to afford compounds of the general formula A.2.

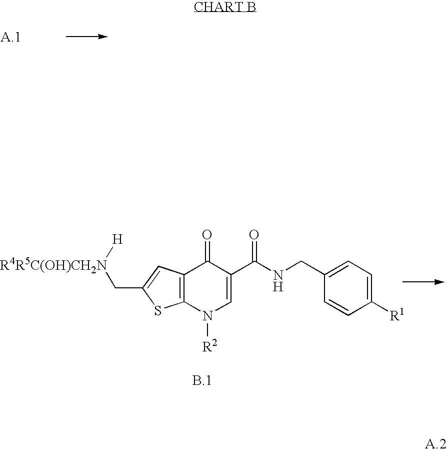

Alternatively, compounds of Formula (I) are prepared as described in Chart C. Compounds of the formula A.1 in which X is a leaving group (e.g. mesylate, chloride, or bromide) are treated with an alkyl primary amine (e.g. methylamine or ethylamine) in the presence of a non-nucleophilic base (e.g. diisopropylethylamine) in a polar solvent (e.g. DMF) to afford products of the formula C.1. The resulting secondary amine is then treated with an electrophile either of the formula R⁴R⁵C(OH)CH₂X (where X is Cl, Br) in the presence of a non-nucleophilic base (e.g. diisopropylethylamine) in a polar solvent (e.g. DMF) or with an epoxide to afford products of the formula A.2. Alternatively, compounds of the formula C. 1 are alkylated with 2-haloketones of the formula R⁴C(O)CH₂X (where X is Cl, Br) according to Chart D to afford products of the formula D.1. The resulting amino ketones are then reduced with an appropriate achiral or chirally-modified reducing agent (e.g. NaBH or diisopinocamphenylchloroborane) to provide compounds of the formula A.2.

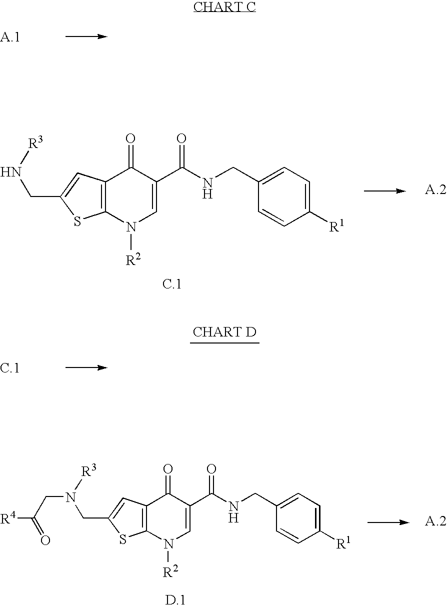

The precursors A. 1 are available from the corresponding alcohols (Y=OH) by treatment with methanesulfonyl chloride in the presence of an organic base (e.g. pyridine or 2,4,6-collidine) and if needed an activating agent (e.g. DMAP), Chart E. Alternatively, compounds of the formula A.1 are available by treatment of a tertiary amino derivative (e.g. Y=N(CH₃)₂ or 4-morpholinyl) with ethyl chloroformate in an appropriate solvent (e.g. chloroform, dichloromethane, 1,2-dichloroethane, or benzene).

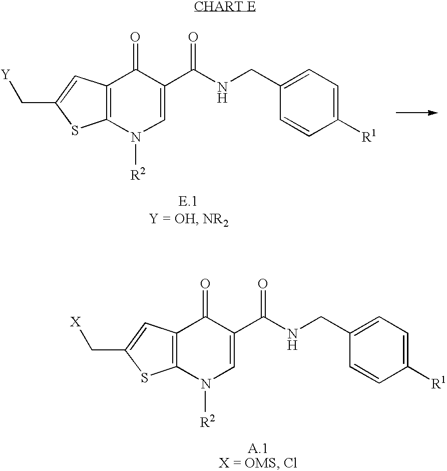

Subsequently, compounds of the general formula E.1 are prepared according to procedures described in U.S. Pat. No. 6,239,142 or exemplified in Charts F, G, and H below.

As described in Chart F, 3-bromo-2-chlorothiophene (F.1) is metalated with lithium diisopropyl amide in tetrahydrofuran at low temperature followed by addition to paraformaldehyde to provide alcohol F.2. The free hydroxyl is protected employing common methodology (Greene, T. W.; Wuts, P. G. M. Protective Groups in Organic Synthesis, 1999) such as the tert-butyldimethylsilyl ether (TBS) by treatment with the corresponding silyl chloride and a weak base (e.g. imidazole) in a polar solvent (e.g. DMF). Metalation of F.3 with n-butyl lithium followed by addition to N-methoxy-N-methylacetamide provides the methyl ketone F.4. Condensation of F.4 with diethyl carbonate in the presence of a strong base (e.g. sodium hydride) affords ketoester F.5. Compound F.5 is then refluxed in a mixture of acetic anhydride and triethylortho-formate to afford an intermediate enol ether which is then condensed with a primary amine or aniline (e.g. R²NH₂) to provide a compound of the formula F.6. The resulting enamines are cyclized by heating in the presence of a base (e.g. sodium hydride, potassium carbonate, or potassium tert-butoxide) in an appropriate solvent (e.g. THF, DMF, or tert-butanol) to provide F.7. Esters of the formula F.7 are converted to amides of the general formula F.8 by either (a) treatment with a substituted benzylamine (e.g. 4-chlorobenzylamine, 4-fluorobenzylamine, or 4-bromobenzylamine) at high temperature or (b) saponification by treatment with an inorganic base such as sodium hydroxide to afford the corresponding carboxylic acid which is then coupled with a substituted benzylamine mediated by 1,1′-carbonyl-diimidazole (or other suitable carboxylic acid activating agent). Subsequent deprotection of the hydroxylprotecting group to afford E.1 is accomplished through common procedures such as treatment with tetrabutylammonium fluoride in the case of silyl ether protection.

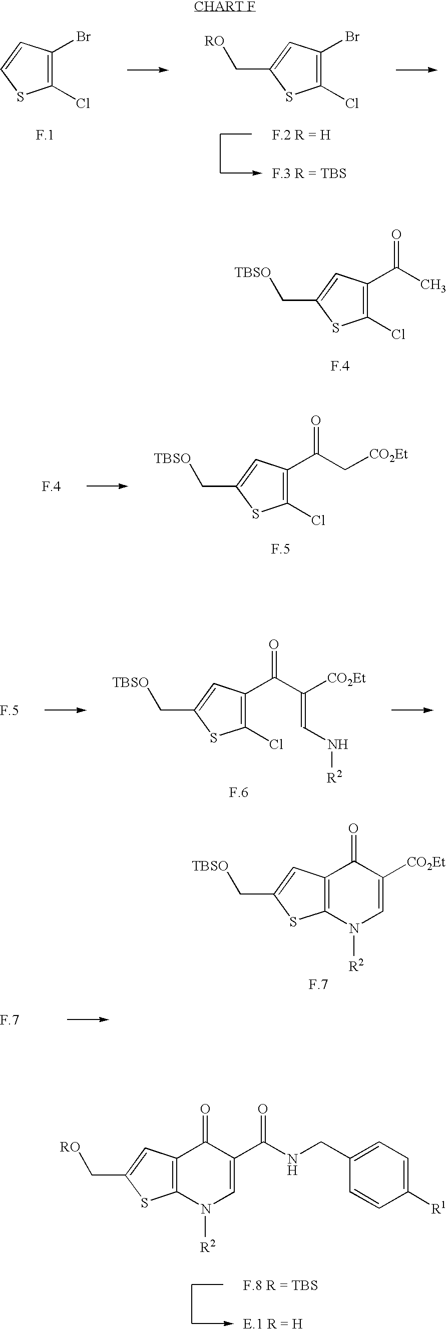

Compounds of formula E.1 (Y=NR₂) may be prepared as described in Chart G. 3-Bromo-2-chlorothiophene (F.1) is metalated with lithium diisopropyl amide in tetrahydrofuran at low temperature and condensed with N,N-dimethylformamide to afford the carboxaldehyde G.1. Reductive amination of G.1 by treating with an amine (e.g. morpholine), acetic acid, and an appropriate reducing agent (e.g. sodium triacetoxyborohydride) affords thiophenes of the formula G.2. Metalation of G.2 with n-butyl lithium followed by addition to N-methoxy-N-methylacetamide provides the methyl ketone G.3. Condensation of G.3 with diethyl carbonate in the presence of a strong base (e.g. sodium hydride) affords ketoester G.4. The resulting ketoester is then treated with a benzylamine (e.g. 4-chlorobenzylamine, 4-fluorobenzylamine, or 4-bromobenzylamine) in refluxing xylene to provide ketoamides of the formula G. 5. Compound G.5 is then refluxed in a mixture of acetic anhydride and triethylortho-formate to afford an intermediate enol ether which is then condensed with a primary amine or aniline (e.g. R²NH₂) to provide a compound of the formula G.6. The resulting enamines are cyclized by heating in the presence of a base (e.g. sodium hydride, potassium carbonate, or potassium tert-butoxide) in an appropriate solvent (e.g. THF, DMF, or tert-butanol).

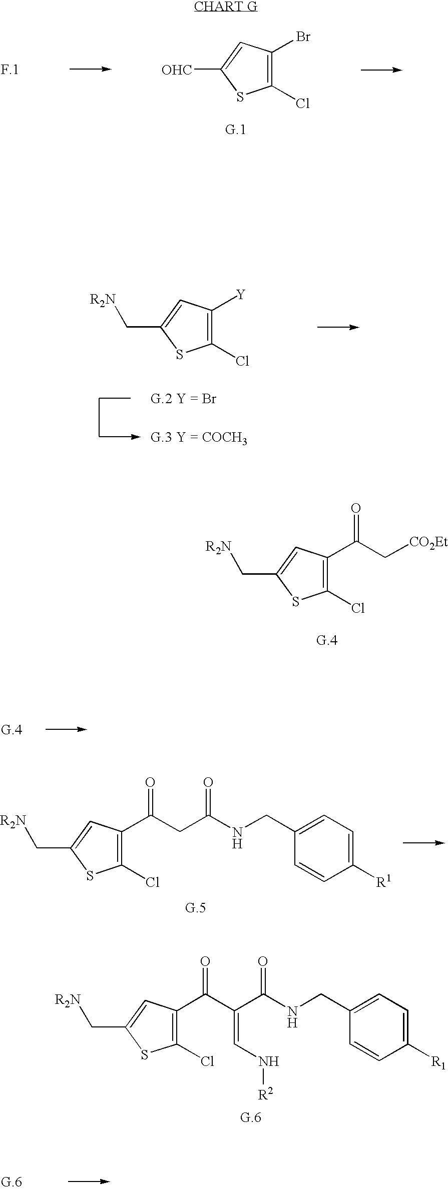

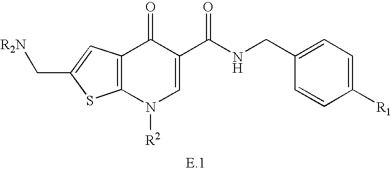

Alternatively, compounds of formula E. 1 (Y=OH) may be prepared as described in Chart H. Ethyl 4-hydroxythieno[2,3-b]pyridine-5-carboxylate (J. Heterocyclic Chem. 1977, 14, 807) is metalated with from two to six equivalents of lithium diisopropylamide at low temperature and is then reacted with dimethylformamide to provide compound H.2. Treatment of H.2 with an appropriate reducing agent (e.g. NaBH₄) in a polar solvent (e.g. ethanol) affords the alcohol H.3. The resulting ester is then reacted with a substituted benzylamine (e.g. 4-chlorobenzylamine, 4-fluorobenzylamine, or 4-bromobenzylamine) at high temperature or under other common amide forming conditions well known to those skilled in the art to provide compounds of the formula H.4. Compound H.4 is alkylated at the ring nitrogen by treatment with an optionally substituted alkyl halide or alkyl sulfonate ester in the presence of a base (e.g. potassium carbonate) or by reaction with an optionally substituted alkanol under Mitsunobu conditions to afford compounds of the general formula E.1. Specific examples of such alkyl halides used in this reaction include but are not limited to iodomethane, iodoethane, 1-iodopropane, 1-iodobutane, and 1-bromo-2-methoxyethane. It would be understood by those skilled in the art that in some cases transient protection of hydroxyl functionality present in the R²X (X=halo or sulfonate) or R²OH reagent used in the above step may be required to facilitate the coupling described in Chart H or subsequent chemistry described in Charts A-E. Specific examples of such protected-hydroxyalkyl halides used in this reaction include but are not limited to 2-(2-bromoethoxy)tetrahydro-2H-pyran, 2-(2-iodoethoxy)tetrahydro-2H-pyran, 2-(3-bromopropoxy)tetrahydro-2H-pyran, 2-(3-iodopropoxy)tetrahydro-2H-pyran, 4-(bromomethyl)-2,2-dimethyl-1,3-dioxolane, 2-(2-(2-chloroethoxy)ethoxy)tetrahydro-2H-pyran, and 2-(chloromethoxy)ethyl benzoate. Procedures to deprotect these cases at the final or intermediate stage are well established (Greene, T. W.; Wuts, P. G. M. Protective Groups in Organic Synthesis, 1999).

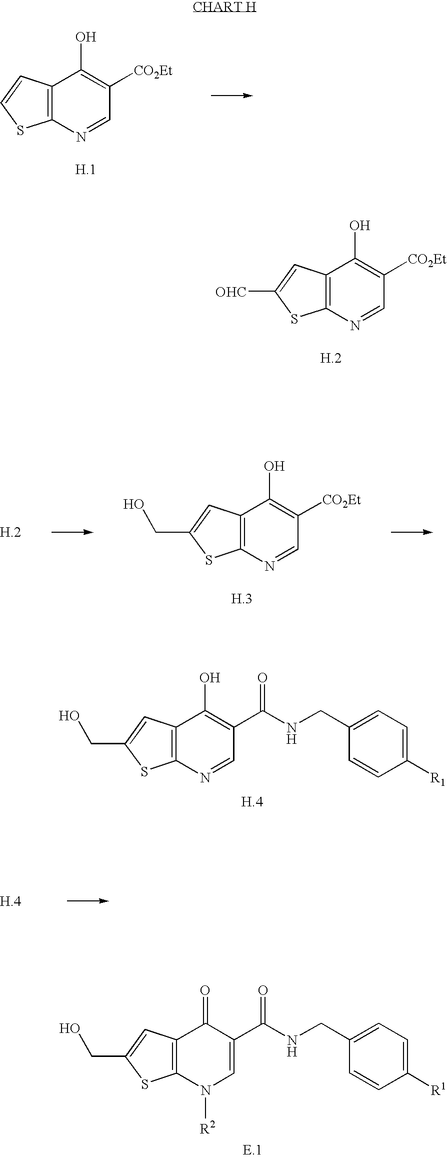

The amine R⁴R⁵C(OH)CH₂NH(R³) in Chart A may be commercially available, can be prepared by procedures know to those skilled in the art, or can be prepared by methods illustrated in Charts I-O. As shown in Chart I, commercially available methylketones I.1 can be halogenated (X=Cl, Br) to provide the haloketones of the formula I.2. The resulting haloketones can be reduced to yield the corresponding halohydrins I.3 employing either achiral (e.g. NaBH₄/CeCl₃) or chiral reduction conditions (e.g. Haniada, T.; Torii, T.; Izawa, K; Noyori, R.; Ikariya, T. Org. Lett. 2002, 4, 4373-4376). The resulting halohydrin is then treated with a primary amine (e.g. methylaniine or ethylamine) to afford amines of the formula I.5. Alternatively, the haloketones can be treated directly with the primary amine (e.g. methylamine or ethylamine) to provide an aminoketone I.4 which can then be reduced under achiral or chiral reduction conditions (Ohkuma, T.; Ishii, D.; Takeno, H.; Noyori, R. J. Am. Chem. Soc. 2000, 122, 6510-6511; Kawamoto, A.; Wills, M. Tetrahedron: Asymmetry 2000, 11, 3257-3261) to afford compounds of the formula I.5. In this case, the basic nitrogen may require transient protection (e.g. tert-butylcarbamate) to facilitate the reduction. The precursor Af-Boc aminoketones J.2 may be prepared as described in Chart J in which a Weinreb amide derivative (Y=N(CH₃)(OCH₃), prepared by methods well know in the literature, e.g. Sibi, M. Org. Prep. Proc. Int. 1993, 25, 15-40) is reacted with metalated tert-butyl dimethylcarbamate in the presence of tetramethylethylenediamine at low temperature. Other compounds of the formula J.1 which also undergo this reaction include carboxamides wherein Y=4-morpholine and thiol esters (e.g. Y=SPh).

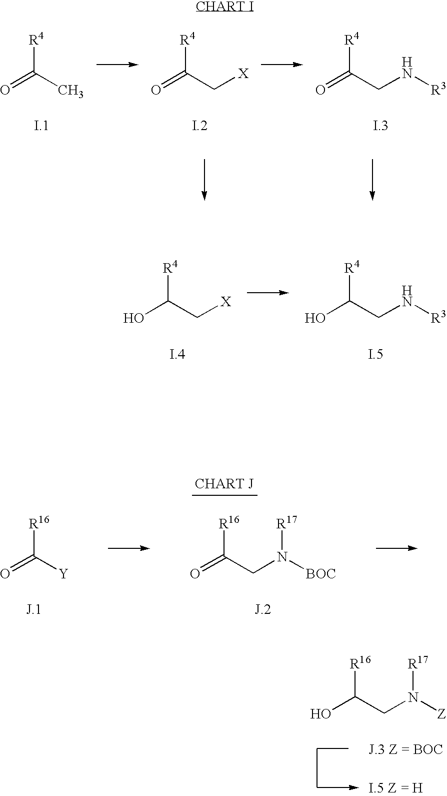

Alternatively, as shown in Chart K specific amines of the formula R⁴R⁵C(OH)CH₂NH(R³) can be prepared from carboxaldehydes K.1 which are commercially available or prepared by methods known to those skilled in the art. Epoxidation of K. 1 with a sulfonium ylide (e.g. trimethylsulfonium iodide) affords epoxides of the formula K.2. Treatment of the epoxides with a primary amine (e.g. methylamine or ethylamine) provides compounds of the formula I.5.

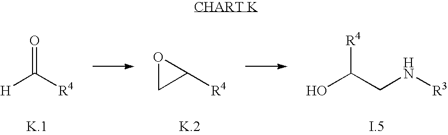

As shown in Chart L, specific amines of the formula R⁴R⁵C(OH)CH₂NH(R³) are also prepared from carbonyl derivatives L. 1 by the reaction with metalated tert-butyl dimethylcarbamate in the presence of tetramethylenediamine at low temperature to afford the BOC-protected amino alcohol L.2. Subsequent cleavage under acidic conditions (e.g. trifluoroacetic acid or hydrochloric acid) or oxazolidinone cyclization under basic conditions (e.g. sodium hydride) followed by basic hydrolysis provides compounds of the formula L.3. In cases where R⁵ is hydroxymethyl, 2-hydroxyethyl, or 1-hydroxyethyl, the hydroxyl group is transiently protected using common protecting groups (Greene, T. W.; Wuts, P. G. M. Protective Groups in Organic Synthesis, 1999) and then deprotected either prior to or after coupling as described in Chart A.

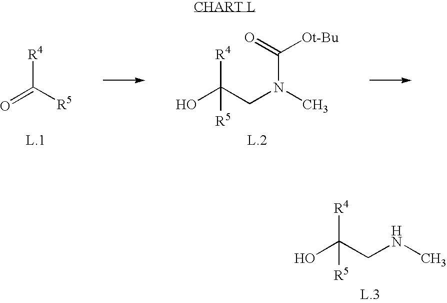

As shown in Chart M, specific amines of the formula R⁴R⁵C(OH)CH₂NH(R³) are also prepared from a protected form of methylaminoacetaldehyde or methylamino-acetaldehyde (e.g. (methyl(trityl)amino)acetaldehyde) (M.1). Treatment of M.1 with a metalated heteroaryl reagent at low temperature affords alcohols of the formula M.2. Subsequent deprotection of the nitrogen protecting group (e.g. in the case of trityl, treatment with an inorganic acid in etheral solution) provides amines of the formula I.5. It would be understood by those skilled in the art that in some cases transient protection of Lewis basic or acidic functionality present in the R⁴ substituent may be required to facilitate the metal reagent formation and subsequent addition described in Chart L for which procedures are well established (Greene, T. W.; Wuts, P. G. M. Protective Groups in Organic Synthesis, 1999).

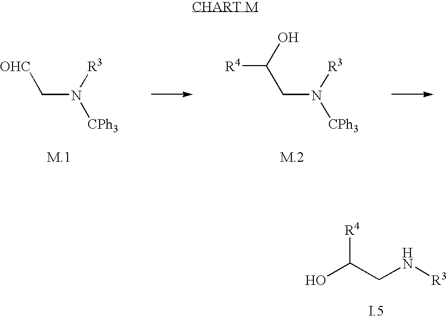

In cases where the R⁵ substituent of the amine R⁴R⁵C(OH)CH₂NH(R³) is methyl or ethyl, the amine may be prepared as described in Chart N. The olefin N. 1 is reacted with N-bromosuccinimide in an ether solvent employing a catalytic amount sulfuric acid to afford the bromohydrin N.2. The resulting bromohydrin is then treated with a primary amine (e.g. methylamine or ethylamine) to afford amines of the formula N.3.

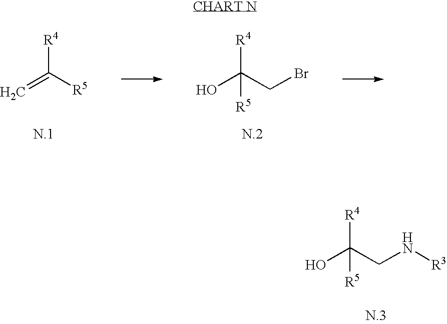

Specific amines of the formula R⁴CH(OH)CH₂NH(CH₃) are also available from primary amines of the formula R⁴CH(OH)CH₂NH₂ according to methods described in Chart O. An amino alcohol of the formula O.1 is treated with dimethyl carbonate and potassium tert-butoxide to afford an oxazoldinone of the formula O.2. The resulting oxazolidinone is subsequently hydrolyzed in the presence of aqueous alkali (e.g. potassium hydroxide) to provide an amino alcohol of the formula O.3.

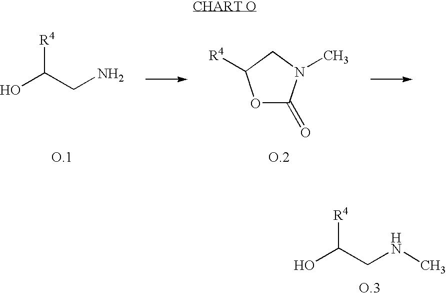

Methods to prepare primary amines of the formula R⁴R⁵C(OH)CH₂NH₂ for use in Chart B are well known to those skilled in the art of organic synthesis (Bergmeier, S. C. Tetrahedron 2000, 56, 2561-2576). In addition to those described herein, representative synthetic examples include 2-amino-1-quinolin-3-ylethanol (Zymalkowski, F.; Tinapp, P. Justus Liebigs Ann. Chem. 1966, 699, 98); 2-amino-1-quinolin-4-ylethanol (Eiter, K; Mrazek, E. Monatsh. Chem. 1952, 83, 915); 2-amino-1-pyridin-4-ylethanol, 2-amino-1-pyridin-3-ylethanol, and 2-amino-1-pyridin-2-ylethanol (Burrus, H.; Powell, G. J. Am. Chem. Soc. 1945, 67, 1468).

The compounds of Formula (I) may be prepared as single enantiomer or as a mixture of individual enantiomers which includes racemic mixtures. Methods to obtain preferentially a single enantiomer from a mixture of individual enantiomers or a racemic mixture are well known to those ordinarily skilled in the art of organic chemistry. Such methods include but are not limited to preferential crystallization of diastereomeric salts (e.g. tartrate or camphor sulfonate), covalent derivatization by a chiral, non-racemic reagent followed by separation of the resulting diastereomers by common methods (e.g. crystallization, chromatographic separation, or distillation) and chemical reversion to scalemic compound, Simulated Moving Bed technology, or high/medium-pressure liquid chromatography employing a chiral stationary phase (Eliel, E. L. Stereochemistry of Organic Compounds, 1994; Subramanian, G. Chiral Separation Techniques: A Practical Approach, 2001). These techniques may be performed on the final compounds of Formula (I) or on any intermediates to compounds of Formula (I) which bear a stereogenic center. Also, to facilitate separation by any of the methods described above, the compounds of Formula (I) or any intermediates to the compounds of Formula (I) which bear a stereogenic center may be transiently reacted with an achiral reagent, separated, and then reverted to scalemic compound by standard synthetic techniques.

It will be apparent to those skilled in the art that the described synthetic procedures are merely representative in nature and alternative synthetic processes are known to one of ordinary skill in organic chemistry.

The compounds of the present invention and pharmaceutically acceptable salts thereof are useful as antiviral agents. Thus, these compounds are useful to combat viral infections in mammals. Specifically, these compounds have anti-viral activity against the herpes virus, cytomegalovirus (CMV). These compounds are also active against other herpes viruses, such as the varicella zoster virus, the Epstein-Barr virus, the herpes simplex virus, and the human herpes virus type 8 (HHV-8).

The compounds of the present invention may also useful for the treatment of several cardiovascular diseases such as atherosclerosis and restenosis. These diseases have been connected with inflammation of coronary vessel walls resulting from infection or reactivation of herpesviruses.

The compounds of the present invention may also be useful for the treatment of herpesvirus infections in animals, for example, illnesses caused by bovine herpesvirus 1-5 (BHV), ovine herpesvirus 1 and 2, Canine herpesvirus 1, equine herpesvirus 1-8 (EHV), feline herpesvirus 1 (FHV), and pseudorabies virus (PRV).

Pharmaceutical Salts

The compound of formula I may be used in its native form or as a salt. In cases where forming a stable nontoxic salt is desired, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, hydrobromide, sulfate, nitrate, bicarbonate, and carbonate salts.

Pharmaceutically acceptable salts may be obtained using standard procedures well known in the art, for example by reacting a compound of the invention with a suitable acid affording a physiologically acceptable anion.

Routes of Administration

In therapeutic use for treating, or combating, viral infections in a mammal (i.e. human and animals) a compound of the present invention, its pharmaceutical compositions and other antiviral agents can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injection, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours or for up to several days.

Dosage

Pharmaceutical compositions suitable for use in the present invention include compositions wherein the active ingredients are contained in an amount sufficient to achieve the intended purpose, i.e., the treatment or prevention of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, an antiviral effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the viral infection being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### Biological Data

While many of the compounds of the present invention have shown activity against the CMV polymerase, these compounds may be active against the cytomegalovirus by this or other mechanisms of action. Thus, the description below of these compounds' activity against the CMV polymerase is not meant to limit the present invention to a specific mechanism of action.

The compounds of the present invention have shown activity in one or more of the assays described below. All of these assays are indicative of a compound's activity and thus of its use as an anti-viral agent.

The HCMV polymerase assay is performed using a scintillation proximity assay (SPA) as described in several references, such as N. D. Cook, et al., Pharmaceutical Manufacturing International, pages 49-53 (1992); K Takeuchi, Laboratory Practice, September issue (1992); U.S. Pat. No. 4,568,649 (1986); which are incorporated by reference herein. Reactions are performed in 96-well plates. The assay is conducted in 100 μl volume with 5.4 mM HEPES (pH 7.5), 11.7 mM KCl, 4.5 mM MgCl₂, 0.36 mg/ml BSA, and 90 nM ³H-dTTP. Assays are run with and without CHAPS, (3-[(3-Cholamidopropyl)-dimethylammonio]-1-propane-sulfonate) at a final concentration of 2 mM. HCMV polymerase is diluted in enzyme dilution buffer containing 50% glycerol, 250 mM NaCl, 10 mM HEPES (pH 7.5), 100 μg/ml BSA, and 0.01% sodium azide. The HCMV polymerase, which is expressed in, recombinant baculovirus-infected SF-9 cells and purified according to literature procedures, is added at 10% (or 10 μl) of the final reaction volume, i.e., 100 μl. Compounds are diluted in 50% DMSO and 10 μl are added to each well. Control wells contain an equivalent concentration of DMSO. Unless noted otherwise, reactions are initiated via the addition of 6 nM biotinylated poly(dA)-oligo(dT) template/primer to reaction mixtures containing the enzyme, substrate, and compounds of interest. Plates are incubated in a 25° C. or 37° C. H₂O bath and terminated via the addition of 40 μl/reaction of 0.5 M EDTA (pH 8) per well. Reactions are terminated within the time-frame during which substrate incorporation is linear and varied depending upon the enzyme and conditions used, i.e., 30 min. for HCMV polymerase. Ten (10) 10 μL of streptavidin-SPA beads (20 mg/ml in PBS/10% glycerol) are added following termination of the reaction. Plates are incubated 10 min. at 37° C., then equilibrated to room temperature, and counted on a Packard Topcount. Linear regressions are performed and IC₅₀'s are calculated using computer software.

A modified version of the above HCMV polymerase assay is performed as described above, but with the following changes: Compounds are diluted in 100% DMSO until final dilution into assay buffer. In the previous assay, compounds are diluted in 50% DMSO. 4.5 mM Dithiothreitol (DTT) is added to the polymerase buffer. Also, a different lot of CMV polymerase is used, which appears to be more active resulting in a more rapid polymerase reaction.

Results of the testing of compounds of the present invention in this assay are shown in Tables 1 below.

All results are listed as Polymerase IC₅₀ (EM) values. In Table 1, the term “n.d.” refers to activity data not determined.

TABLE 1

| | Polymerase IC₅₀ (μM) | | |
|---|---|---|---|
| Example | HCMV | HSV | VZV |
| 1 | 0.16 | 0.58 | 0.22 |
| 2 | 0.08 | 0.35 | 0.13 |
| 3 | 0.20 | 0.47 | 0.22 |
| 4 | 0.09 | 0.10 | 0.03 |
| 5 | 0.06 | 0.06 | 0.02 |
| 6 | 0.62 | n.d. | n.d. |
| 7 | 0.07 | 0.15 | 0.04 |
| 8 | 0.13 | 0.15 | 0.05 |
| 9 | 0.05 | 0.13 | 0.03 |
| 10 | 0.11 | 0.10 | 0.05 |
| 11 | 0.04 | 0.08 | 0.02 |
| 12 | 0.31 | 0.84 | 0.23 |
| 13 | 0.37 | 0.55 | 0.17 |
| 14 | 1.20 | n.d. | n.d. |
| 15 | 1.07 | n.d. | n.d. |
| 16 | 0.31 | 1.09 | 0.21 |
| 17 | 2.19 | n.d. | n.d. |
| 18 | 1.30 | n.d. | n.d. |
| 19 | 1.12 | n.d. | n.d. |
| 20 | 1.16 | n.d. | n.d. |
| 21 | 1.03 | n.d. | n.d. |
| 22 | 0.58 | n.d. | n.d. |
| 23 | 0.28 | 0.49 | 0.11 |
| 24 | 0.23 | n.d. | n.d. |
| 25 | 0.15 | n.d. | n.d. |

### EXAMPLES

Preparation 1.

N-(4-Chlorobenzyl)-2-(chloromethyl)-7-methyl-4-oxo-4,7-dihydrothieno[2,3-b]-pyridine-5-carboxamide.

Procedure A. N-(4-Chlorobenzyl)-2-(hydroxymethyl)-7-methyl-4-oxo-4,7-dihydrothieno[2,3-b]pyridine-5-carboxamide (3.00 g, prepared as described in U.S. Pat. No. 6,239,142) is dissolved in DMF (150 mL). DMAP (0.150 g), 2,4,6-collidine (2.73 mL), and methanesulfonyl chloride (1.60 mL) are added, and the reaction mixture is stirred at room temperature for 18 h. The reaction mixture is poured into water (300 mL). The resulting pale yellow solid is filtered off and triturated with acetonitrile to yield 2.75 g of the title compound. Physical characteristics. M.p. 250-256° C. (dec); ¹H NMR (400 MHz, DMSO-d₆) δ 10.48, 8.74, 7.58, 7.41-7.33, 5.16, 4.55, 3.97; ¹³C NMR (DMSO-d₆) δ 172.5, 164.5, 151.8, 146.4, 138.9, 135.7, 131.7, 130.5, 129.5, 128.7, 124.0, 115.0, 43.4, 41.8, 41.1; MS (EI) m/z 380 (M⁺); HRMS (FAB) m/z 381.0255 (M+H)⁺. Anal. Found: C, 53.34; H, 3.70; N, 7.30; Cl, 17.91; S, 8.51.

Procedure B. A 25 mL round-bottomed flask is charged with N-(4-chlorobenzyl)-7-methyl-2-(morpholin-4-ylmethyl)-4-oxo-4,7-dihydrothieno[2,3-b]pyridine-5-carboxamide (1.00 g, prepared as described in U.S. Pat. No. 6,239,142) and chloroform (10 ml) via syringe. Ethyl chloroformate (0.55 mL) is added via syringe with stirring under nitrogen. The slurry is heated to reflux overnight. Anhydrous diethyl ether (10 ml) is added to the slurry with stirring under nitrogen. The solid is filtered and washed with diethyl ether (3×10 mL). The product is dried in the vacuum oven at 40° C. to afford 0.93 g of the title compound as colorless crystals. Physical characteristics. ¹H NMR (400 MHz, TFA-d) δ 9.09, 7.69, 7.22, 4.81, 4.62, 4.27; ¹³C NMR (100 MHz, TFA-d) δ 167.6, 166.6, 156.3, 145.2, 143.6, 134.9, 133.3, 129.1, 129.0, 127.4, 119.6, 109.9, 45.2, 44.0, 38.0. Anal. Found: C, 53.44; H, 3.66; N, 7.35; Cl, 18.29.

Repeating the above experiment in refluxing methylene chloride for 8 hours gave a 95.5% yield of the title compound.

Procedure C. A 100 mL three-necked flask containing N-(4-chlorobenzyl)-2-((dimethylamino)methyl)-7-methyl-4-oxo-4,7-dihydrothieno[2,3-b]pyridine-5-carboxamide (3.00 g) is purged with nitrogen. Dry methylene chloride (34 mL) is added via syringe followed by ethyl chloroformate (1.84 mL). The slurry is heated to reflux for two hours and then allowed to cool and stir overnight. Anhydrous diethyl ether (34 ml) is added and the mixture is stirred for 50 min before filtration and washing with diethyl ether (2×15 mL). The solid is dried in the vacuum oven at 32° C. for 2.5 hr to provide 2.86 g of the title compound. Physical characteristics. Anal. Found: C, 53.45; H, 3.67; N, 7.31; Cl, 18.26.

Preparation 2.

N-(4-Chlorobenzyl)-7-((2,2-dimethyl-1,3-dioxolan-4-yl)methyl)-2-(hydroxymethyl)-4-oxo-4,7-dihydrothieno[2,3-b]pyridine-5-carboxamide.

Cesium carbonate (3.91 g) is added to a solution of N-(4-chlorobenzyl)-4-hydroxy-2-(hydroxymethyl)thieno[2,3-b]pyridine-5-carboxamide (3.49 g, prepared as described in U.S. Pat. No. 6,239,142) and 4-(bromomethyl)-2,2-dimethyl-1,3-dioxolane (1.95 g) in DMF (20 mDL). The reaction mixture is stirred at 100° C. for 17 h. The solvent is evaporated and the residue is dissolved in 10% CH₃OH in CH₂Cl₂. The mixture is washed with water and the organic layer is dried (MgSO₄), filtered, and concentrated. The crude product is crystallized from EtOAc to afford 2.7 g of the title compound as a white solid. Physical characteristics. ¹H NMR (400 MHz, DMSO-d₆) δ 10.53, 8.70, 7.40, 7.34, 7.28, 5.79, 4.69, 4.53, 4.50, 4.30, 4.14, 3.77, 1.34, 1.23; MS (EI) m/z 462 (N); HRMS (FAB) m/z 463.1087 (M+H)⁺. Anal. Found: C, 57.07; H, 5.01; N, 6.05.

Preparation 3.

N-(4-Chlorobenzyl)-7-methyl-2-((methylamino)methyl)-4-oxo-4,7-dihydro-thieno[2,3-b]pyridine-5-carboxamide.

N-(4-Chlorobenzyl)-2-(chloromethyl)-7-methyl-4-oxo-4,7-dihydrothieno[2,3-b]pyridine-5-carboxamide (Preparation 1, 2.00 g) is suspended in DMF (120 mL), and a 2.0 M solution of methylamine in THF (27 mL) is added. The reaction mixture is heated to 70° C. for 1 h. The reaction is allowed to cool to room temperature and is poured into water (350 mL). The resulting solid is filtered and purified by column chromatography (CH₂Cl₂/methanol; 98/2, 95/5) to yield 1.07 g of the title compound as a white solid. Physical characteristics. M.p. 196-199° C.; ¹H NMR (400 MHz, DMSO-d₆) δ 10.62, 8.69, 7.41-7.31, 4.55, 3.95, 3.88, 2.30; MS (ESI+) m/z 376 (M+H)⁺, Anal. Found: C, 57.30; H, 4.86; N, 11.06; Cl, 9.23; S, 8.28.

Preparation 4.

2-(Chloromethyl)-N-((4-chlorophenyl)methyl)-7-((2,2-dimethyl-1,3-dioxolan-4-yl)methyl]-4,7-dihydro-4-oxothieno[2,3-b]pyridine-5-carboxamide.

2,4,6-Collidine (1.78 mL) and a few crystals of 4-N,N-dimethylaminopyridine are added to a solution of N-(4-chlorobenzyl)-7-((2,2-dimethyl-1,3-dioxolan-4-yl)methyl)-2-(hydroxymethyl)-4-oxo-4,7-dihydrothieno[2,3-b]pyridine-5-carboxamide (Preparation 2, 2.33 g) in DMF (15 m]L). Methanesulfonyl chloride (0.93 mL) is added drop-wise and the reaction is stirred at room temperature for 4 hours. The solvent is evaporated and the residue is dissolved in 10% MeOH in CH₂Cl₂. The mixture is washed with water, dried (MgSO₄), filtered, and concentrated. The residue is purified by column chromatography (CH₂Cl₂/methanol, 95/5). The crude product is crystallized from EtOAc, filtered and washed with diethyl ether to afford 1.73 g of the title compound as white crystals. Physical characteristics: ¹H NMR (400 MHz, DMSO-d₆) δ 10.42, 8.73, 7.55, 7.39, 7.34, 5.15, 4.54, 4.51, 4.30, 4.14, 3.77, 1.34, 1.23; HRMS (FAB) m/z 481.0758 (M+H)⁺. Anal. Found: C, 55.18; H, 4.76; N, 5.66.

Preparation 5.

N-(4-Chlorobenzyl)-2-(hydroxymethyl)-4-oxo-7-(3-(tetrahydro-2H-pyran-2-yloxy)propyl)-4,7-dihydrothieno[2,3-b]pyridine-5-carboxamide.

Cesium carbonate (5.54 g) is added to a solution of N-(4-chlorobenzyl)-4-hydroxy-2-(hydroxymethyl)thieno[2,3-b]pyridine-5-carboxamide (5.23 g, prepared as described in U.S. Pat. No. 6,239,142) and 2-(3-iodopropoxy)tetrahydro-2H-pyran (4.32 g, prepared by mixing equal molar amounts of 2-iodopropanol and 3,4-dihydro-2H-pyran) in DMF (20 mL). The mixture is heated at 60° C. for 4 hours. The solvent is evaporated and the residue is dissolved in 10% MeOH in CH₂Cl₂. The mixture is washed with water and the organic layer is dried (MgSO₄), filtered, and concentrated. The crude product is purified by column chromatography (CH₂Cl₂/methanol, 95/5) followed by recrystallization from EtOAc to afford 4.82 g of the title compound as white crystals. Physical characteristics. ¹H NMR (400 MHz, DMSO-d₆) δ 10.55, 8.71, 7.39, 7.33, 7.29, 5.79, 4.70, 4.53, 4.49, 4.38, 3.68, 3.37, 2.11, 1.63, 1.53, 1.40; MS (EI) m/z 490 (M⁺); Anal. Found: C, 58.74; H, 5.66; N, 5.61.

Preparation 6.

N-(4-Chlorobenzyl)-2-(chloromethyl)-4-oxo-7-(3-(tetrahydro-2H-pyran-2-yloxy)-propyl)-4,7-dihydrothieno[2,3-b]pyridine-5-carboxamide.

2,4,6-Collidine (2.51 mL) and a few crystals of 4-N,N-dimethylaminopyridine is added to a solution of N-(4-chlorobenzyl)-2-(hydroxymethyl)-4-oxo-7-(3-(tetrahydro-2H-pyran-2-yloxy)propyl)-4,7-dihydrothieno[2,3-b]pyridine-5-carboxamide (Preparation 5, 4.0 g) in DMF (20 mL). Methanesulfonyl chloride (1.38 mL) is added drop-wise and the reaction is stirred at 60° C. for 5 hours. The solvent is evaporated and the residue dissolved in 10% MeOH in CH₂Cl₂. The mixture is washed with water and the organic layer is dried (MgSO₄), filtered, and concentrated. The residue is purified by column chromatography (CH₂Cl₂methanol, 95/5). The crude product is crystallized from EtOAc, filtered, and washed with diethyl ether to afford 2.35 g of the title compound as white crystals. Physical characteristics. ¹H NMR (400 MHz, DMSO-d₆) δ 10.43, 8.74, 7.56, 7.39, 7.34, 5.15, 4.54, 4.38, 3.70, 3.38, 2.11, 1.61, 1.51, 1.38; MS (EI) m/z 508 (M⁺); HRMS (FAB) m/z 509.1064 (M+H)⁺. Anal. Found: C, 56.00; H, 5.11; N, 5.56.

Preparation 7.

N-(4-Chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide.

Cesium carbonate (3.91 g) is added to a solution of N-(4-chlorobenzyl)-4-hydroxy-2-(hydroxymethyl)thieno[2,3-b]pyridine-5-carboxamide (3.49 g, prepared as described in U.S. Pat. No. 6,239,142) and 2-(2-iodoethoxy)tetrahydro-2H-pyran (2.56 g, prepared by mixing equal molar amounts of 2-iodoethanol and 3,4-dihydro-2H-pyran) in DMF (20 mL). The reaction mixture is stirred at 100° C. for 17 hours. The solvent is evaporated and the residue is dissolved in 10% CH₃OH in CH₂Cl₂. The mixture is washed with water and the organic layer is dried (MgSO₄), filtered, concentrated. The crude product is crystallized from EtOAc to afford 3.8 g of the title compound as a white solid. Physical characteristics. ¹H NMR (400 MHz, DMSO-d₆) δ 10.59, 8.71, 7.39, 7.38, 7.29, 5.79, 4.69, 4.58, 4.54, 4.48, 3.96, 3.78, 3.30, 1.54, 1.39, 1.29; MS (EI) m/z 476 (M⁺); HRMS (FAB) m/z 477.1245 (M+H)⁺.

Preparation 8.

N-(4-Chlorobenzyl)-2-(chloromethyl)-4-oxo-7-(2-(tetrahydro-2H-pyran-2-yloxy)ethyl)]-4,7-dihydrothieno[2,3-b]pyridine-5-carboxamide.

2,4,6-Collidine (2.9 μL) and a few crystals of 4-N,N-dimethylaminopyridine is added to a solution of N-(4-chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide (Preparation 7, 3.5 g) in DMF (20 mL). Methanesulfonyl chloride (1.7 mL) is added drop-wise and the reaction mixture is stirred at room temperature for 72 h. The reaction mixture is poured into water (100 mL) and filtered. The filtrate is extracted with 10% MeOH in CH₂Cl₂. The organic layer is dried (MgSO₄), filtered, and concentrated to afford 2.8 g of the title compound as a white solid. Physical characteristics. ¹H NMR (400 MHz, DMSO-d₆) δ 10.43, 8.75, 7.55, 7.38, 7.33, 5.14, 4.59, 4.53, 4.49, 3.96, 3.79, 3.29, 1.52, 1.38, 1.28; MS (EI) m/z 494 (M⁺); FRMS (FAB) m/z 495.0904 (M+H)⁺.

Preparation 9.

N-(4-Chlorobenzyl)-2-(chloromethyl)-7-(2-hydroxyethyl)]-4-oxo-4,7-dihydrothieno[2,3-b]pyridine-5-carboxamide.

2,4,6-Collidine (2.9 mL) and a few crystals of 4-N,N-dimethylaminopyridine is added to a solution of N-(4-chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide (Preparation 7, 3.5 g) in DMF (20 mL). Methanesulfonyl chloride (1.7 mL) is added drop-wise and the reaction mixture is stirred at room temperature for 72 h. The reaction mixture is poured into water (100 mL) and filtered. The solid is recrystallized from acetonitrile to afford 1.27 g of the title compound as a white solid. Physical characteristics. ¹H NMR (400 MHz, DMSO-d₆) δ 10.47, 8.67, 7.55, 7.40, 7.34, 5.15, 5.14, 4.54, 3.34, 2.51; MS (EI) m/z 410 (M⁺); HRMS (FAB) m/z 411.0332 (M+H)⁺. Anal. Found: C, 52.27; H, 4.05; N, 6.93.

Preparation 10.

N-(4-Chlorobenzyl)-7-ethyl-2-(hydroxymethyl)-4-oxo-4,7-dihydrothieno[2,3-b]-pyridine-5-carboxamide.

Potassium carbonate (0.87 g) and iodoethane (0.5 mL) are added to a solution of N-(4-chlorobenzyl)-4-hydroxy-2-(hydroxymethyl)thieno[2,3-b]pyridine-5-carboxamide (2.0 g, prepared as described in U.S. Pat. No. 6,239,142) in anhydrous DMF (60 mL). The reaction mixture is stirred at room temperature for 18 h. The mixture is diluted with water (150 mL) and filtered. The resulting white powder is washed with water (15 mL) followed by diethyl ether (15 mL) and dried in a vacuum oven to afford 1.64 g of the title compound as a white solid. Physical characteristics. M.p. 169-172° C.; ¹H NMR (300 MHz, DMSO-d₆) δ 10.65, 8.74, 7.37, 7.29, 5.81, 4.70, 4.54, 4.32, 1.44; HRMS (FAB) m/z 377.0720 (M+H)⁺. Anal. Found: C, 56.87; H, 4.77; N, 7.38; Cl, 9.35; S, 8.44.

Preparation 11.

N-(4-Chlorobenzyl)-2-(chloromethyl)-7-ethyl-4-oxo-4,7-dihydrothieno[2,3-b]-pyridine-5-carboxamide.

4-N,N-Dimethylaminopyridine (80 mg), 2,4,6-collidine (1.41 mL), and methanesulfonyl chloride (0.83 mL) are added to a solution of N-(4-chlorobenzyl)-7-ethyl-2-(hydroxymethyl)-4-oxo-4,7-dihydrothieno[2,3-b]pyridine-5-carboxamide (Preparation 10, 1.61 g) in anhydrous DMF (80 mL). The reaction mixture is stirred at room temperature for 24 h. The mixture is diluted with water (150 mL) and filtered. The resulting white powder is recrystallized from acetonitrile and dried in a vacuum oven to afford 1.4 g of the title compound as a white solid. Physical characteristics. M.p. 199-200° C.; ¹H NMR (300 MHz, DMSO-d₆) δ 10.45, 8.77, 7.57, 7.38, 5.15, 4.54, 4.32, 1.44. Anal. Found: C, 54.53; H, 3.94; N, 7.03; Cl, 17.57; S, 8.09.

Preparation 12.

N-(4-Chlorobenzyl)-7-propyl-2-(hydroxymethyl)-4-oxo-4,7-dihydrothieno[2,3-b]-pyridine-5-carboxamide.

Potassium carbonate (0.91 g) and 1-iodopropane (0.64 mL) are added to a solution of N-(4-chlorobenzyl)-4-hydroxy-2-(hydroxymethyl)thieno[2,3-h]pyridine-5-carboxamide (2.0 g, prepared as described in U.S. Pat. No. 6,239,142) in anhydrous DMF (60 mL). The reaction mixture is stirred at room temperature for 4 h. The mixture is diluted with water (150 mL) and filtered. The resulting white powder is washed with water (15 mL) followed by diethyl ether (15 mL) and dried in a vacuum oven to afford 1.73 g of the title compound as a white solid. Physical characteristics. M.p. 174-175° C.; ¹H NMR (300 MHz, DMSO-d₆) δ 10.62, 8.72, 7.38, 7.29, 5.80, 4.69, 4.55, 4.27, 1.87, 0.89; Anal. Found: C, 58.20; H, 4.96; N, 7.13; Cl, 8.98; S, 8.16.

Preparation 13.

N-(4-Chlorobenzyl)-2-(chloromethyl)-7-propyl-4-oxo-4,7-dihydrothieno[2,3-b]-pyridine-5-carboxamide.

4-N,N-Dimethylaminopyridine (80 mg), 2,4,6-collidine (1.39 mL), and methanesulfonyl chloride (0.81 mL) are added to a solution of N-(4-chlorobenzyl)-7-propyl-2-(hydroxymethyl)-4-oxo-4,7-dihydrothieno[2,3-b]pyridine-5-carboxamide (Preparation 12, 1.63 g) in anhydrous DMF (80 mL). The reaction mixture is stirred at room temperature for 24 h. The mixture is diluted with water (150 mL) and filtered. The resulting light yellow powder is recrystallized from acetonitrile and dried in a vacuum oven to afford 1.4 g of the title compound as a light yellow solid. Physical characteristics. M.p. 186.5-188° C.; ¹H NMR (300 MHz, DMSO-d₆) δ 10.45, 8.75, 7.56, 7.39, 5.15, 4.54, 4.27, 1.85, 0.91. Anal. Found: C, 55.76; H, 4.59; N, 6.95; Cl, 16.88; S, 7.80.

Preparation 14.

N-(4-Chlorobenzyl)-2-(hydroxymethyl)-7-(2-methoxyethyl)-4-oxo-4,7-dihydrothieno[2,3-b]pyridine-5-carboxamide.

Potassium carbonate (5.0 g) and bromoethylmethyl ether (5.0 g) are added to a solution of N-(4-chlorobenzyl)-4-hydroxy-2-(hydroxymethyl)thieno[2,3-b]pyridine-5-carboxamide (11.4 g, prepared as described in U.S. Pat. No. 6,239,142) in anhydrous DMF (350 mL). The reaction mixture is stirred at room temperature for 18 h. The mixture is diluted with water (600 mL) and filtered. The resulting white powder is dried in a vacuum oven to afford 8.44 g of the title compound as a white solid. Physical characteristics. M.p. 193° C.; ¹H NMR (300 MHz, DMSO-d₆) δ 10.58, 8.65, 7.37, 7.29, 5.82, 4.70, 4.54, 4.47, 3.76, 3.24; HRMS (FAB) m/z 407.0836 (M+H)⁺. Anal. Found: C, 55.81; H, 4.71; N, 6.90; Cl, 8.58; S, 7.81.

Preparation 15.

N-(4-Chlorobenzyl)-2-(chloromethyl)-7-(2-methoxyethyl)-4-oxo-4,7-dihydro-thieno[2,3-b]pyridine-5-carboxamide.

4-N,N-Dimethylaminopyridine (360 mg), 2,4,6-collidine (6.5 mL), and methanesulfonyl chloride (3.8 mL) are added to a solution of N-(4-chlorobenzyl)-2-(hydroxymethyl)-7-(2-methoxyethyl)-4-oxo-4,7-dihydrothieno[2,3-b]pyridine-5-carboxamide (Preparation 14, 8.0 g) in anhydrous DMF (360 mL). The reaction mixture is stirred at room temperature for 18 h. The mixture is diluted with water (600 mL) and filtered. The resulting solid is dried in a vacuum oven to afford 7.03 g of the title compound as an off-white solid. Physical characteristics. M.p. 192-193° C.; ¹H NMR (300 MHz, DMSO-d₆) δ 10.48, 8.67, 7.55, 7.37, 5.14, 4.53, 4.46, 3.74, 3.24; HRMS (FAB) m/z 425.0480 (M+H)⁺. Anal. Found: C, 53.38; H, 4.37; N, 6.66; Cl, 15.77; S, 7.69.

Preparation 16.

N-(4-Chlorobenzyl)-2-(hydroxymethyl)-4-oxo-7-(2-(2-(tetrahydro-2H-pyran-2-yloxy)ethoxy)ethyl)-4,7-dihydrothieno[2,3-b]pyridine-5-carboxamide.

Cesium carbonate (3.25 g) is added to a solution of N-(4-chlorobenzyl)-4-hydroxy-2-(hydroxymethyl)thieno[2,3-b]pyridine-5-carboxamide (3.5 g, prepared as described in U.S. Pat. No. 6,239,142) and 2-(2-(2-chloroethoxy)ethoxy)tetrahydro-2H-pyran (2.1 g) in DMF (12 mL). The mixture is heated at 100° C. for 6 hours. The solvent is evaporated and the residue is purified by column chromatography (CH₂Cl₂/methanol, 95/1) to afford 2.65 g of the title compound as a pale yellow solid. Physical characteristics. ¹H NMR (400 MHz, DMSO-d₆) δ 10.54, 8.66, 7.40-7.28, 5.80, 4.69, 4.53, 4.46, 4.41, 3.85, 3.60, 3.54, 3.48, 3.37, 3.23, 1.51-1.31; MS (CI) m/z 521 (M+H)⁺. Anal Found: C, 57.58; H, 5.61; N, 5.36.

Preparation 17.

N(4-Chlorobenzyl)-2-(chloromethyl)-4-oxo-7-(2-(2-(tetrahydro-2H-pyran-2-yloxy)ethoxy)ethyl)-4,7-dihydrothieno[2,3-b]pyridine-5-carboxamide.

2,4,6-Collidine (1.6 mL) and 4-N,N-dimethylaminopyridine (20 mg) is added to a suspension of N-(4-chlorobenzyl)-2-(hydroxymethyl)-4-oxo-7-(2-(2-(tetrahydro-2H-pyran-2-yloxy)ethoxy)ethyl)-4,7-dihydrothieno[2,3-b]pyridine-5-carboxamide (Preparation 16, 2.62 g) in THF (15 mL). Methanesulfonyl chloride (0.78 mL) is added and the reaction is heated at 50° C. for 1 h. The solvent is evaporated and the residue dissolved in chloroform. The organic layer is washed with water, dried (MgSO₄), and concentrated. The crude product is purified by column chromatography (CH₂Cl₂/methanol, 9515) and then crystallized from EtOAc to afford 1.8 g of the title compound as a tan solid. Physical characteristics. ¹H NMR (400 MHz, DMSO-d₆) δ 10.54, 8.70, 7.55, 7.40-7.33, 5.14, 4.53, 4.47, 4.39, 3.86, 3.61-3.46, 3.37, 3.33, 3.22, 1.49-1.31. Anal. Found: C, 55.65; H, 5.19; N, 5.11.

Preparation 18.

rac-2-(Methylamino)-1-pyridin-3-ylethanol Hydrobromide.

A solution of 3-bromoacetylpyridine hydrobromide (Tsushima, S., et al., EP 278621, 1988) (14.0 g) in methanol (52 mL) is cooled to −10° C. (internal). A solution of sodium borohydride (2.92 g) in water (52 mL) is added drop-wise over 45 min. The reaction mixture is allowed to stir for an additional 5-10 min after the addition is complete. Hydrobromic acid (48%) is added to pH 3-4. The reaction mixture is concentrated in vacuo to remove methanol and then poured into cold ethyl acetate (100 mL)/2 N NaOH (25 mL). The organic layer is removed and the aqueous layer is adjusted to pH 12 with a 2 N NaOH solution. The aqueous layer is extracted with ethyl acetate (2×100 mL). The combined organic layers are dried (MgSO₄), filtered, and concentrated in vacuo to yield 9.098 g of the bromohydrin as a yellow oil. The crude bromohydrin (5.00 g) is dissolved in methanol (20 mL), and a 2.0 M solution of methylamine in methanol (125 mL) is added. The reaction mixture is heated to reflux for 1 h. The reaction mixture is allowed to cool to room temperature and then concentrated in vacuo. The resulting orange oil is crystallized from methanol/ethyl acetate to yield 2.406 g of the title compound as a yellow solid. Physical characteristics. M.p. 146-170° C.; ¹H NMR (400 MHz, DMSO-d₆) δ 8.62-8.61, 8.54-8.53, 8.41, 7.83-7.81, 7.45-7.42, 6.27, 5.02-4.99, 3.23-3.17, 3.13-3.07, 2.61; MS (ESI+) m/z 153 (M+H)⁺.

Preparation 19.

rac-2-(Methylamino)-1-pyridin-4-ylethanol.

A solution of 4-bromoacetylpyridine hydrobromide (Taurins, A.; Blaga, A. J. Heterocyclic Chem., 1970, 7, 1137-1141) (14.5 g) in methanol (150 mL) is cooled to −10° C. (internal). A solution of sodium borohydride (3.03 g) in water (50 mL) is added drop-wise over 1 h. The reaction mixture is allowed to stir for an additional 5-10 min after the addition is complete. Hydrobromic acid (48%) is added to pH 3-4. The reaction mixture is concentrated in vacuo to remove methanol and then poured into cold ethyl acetate (100 mL)/2 N NaOH (50 mL). The organic layer is removed, dried (MgSO₄), filtered, and concentrated in vacuo to yield 8.406 g of the bromohydrin as a pink solid. The crude bromohydrin (5.00 g) is dissolved in methanol (20 mL), and a 2.0 M solution of methylamine in methanol (125 mL) is added. The reaction mixture is stirred at room temperature for 18 h and then concentrated in vacuo. The resulting orange oil is dissolved in water (50 mL), adjusted to pH 12 with a 2 N NaOH solution, and extracted with ethyl acetate (4×100 mL). The combined organic layers are dried (MgSO₄), filtered, and concentrated in vacuo. The resulting yellow solid is purified by column chromatography (CHCl₃/methanol, 95/5, 90/10; CHCl₃/methanol/NH₄OH, 90/10/1) to yield 0.986 g of the title compound as a pale orange solid. Physical characteristics. M.p. 90-93° C.; ¹H NMR (400 MHz, DMSO-d₆) δ 8.49, 7.35, 5.49, 4.66-4.63, 2.63-2.54, 2.29, 1.67; MS (ESI+) m/z 153 (M+H)⁺.

Preparation 20.

rac-2-(Methylamino)-1-pyridin-2-ylethanol.

Procedure A. A solution of 2-bromoacetylpyridine hydrobromide (Tsushima, S., et al., EP 278621, 1988) (8.87 g) in methanol (90 mL) is cooled to −10° C. (internal). A solution of sodium borohydride (1.85 g) in water (30 mL) is added drop-wise over 1 h. The reaction mixture is allowed to stir for an additional 5-10 min after the addition is complete. Hydrobromic acid (48%) is added to pH 3-4. The reaction mixture is concentrated in vacuo to remove methanol and then poured into cold ethyl acetate (60 mL)/2 N NaOH (30 mL). The organic layer is removed, and the aqueous layer is extracted with ethyl acetate (3×60 mL). The combined organic layers are dried (MgSO₄), filtered, and concentrated in vacuo to yield 5.73 g of the bromohydrin as a yellow oil. The crude bromohydrin (5.00 g) is dissolved in methanol (20 mL), and a 2.0 M solution of methylamine in methanol (125 mL) is added. The reaction mixture is stirred at room temperature for 18 h and then concentrated in vacuo. The resulting orange oil is dissolved in a 2 N NaOH solution (25 mL) and extracted with CH₂Cl₂ (8×100 mL). The combined organic layers are dried (MgSO₄), filtered, and concentrated in vacuo. The resulting orange oily solid is purified via column chromatography (CHCl₃/methanol, 95/5, 90/10; CHCl₃/methanol/NH₄OH, 90/10/1) to yield 1.324 g of the title compound as a yellow oil. Physical characteristics. ¹H NMR (400 MHz, DMSO-d₆) δ 8.48-8.47, 7.79-7.75, 7.50-7.48, 7.25-7.22, 5.44, 4.69-4.66, 2.80-2.76, 2.64-2.59, 2.30; MS (ESI+) m/z 153 (M+H)⁺.

Procedure B. A 3-neck, round-bottomed flask, fitted with mechanical stirring, thermocouple, addition funnel and nitrogen inlet is charged with N-bromosuccinimide (3.72 g) and water (20 mL). The resulting slurry is cooled to between 0-5° C. in an ice/water bath and acetic acid (1.32 g) is added. A solution of 2-vinyl pyridine (2.0 g) in t-butanol (3 mL) is added drop-wise keeping the temperature below 10° C. The mixture is stirred maintaining a temperature below 10° C. for 2 h. A solution of sodium hydroxide (2.7 g) in water (20 mL) is slowly added keeping the temperature below 25° C. The resulting solution is stirred for 1 h and MTBE (20 mL) is added. The aqueous layer is separated and washed with MTBE (10 mL). The combined organic layers are washed with brine and concentrated. The oil is dissolved in THF (4 mL) and the resulting solution is added drop-wise to a 40% aqueous solution of methyl amine (15 g) maintaining the temperature at 10-20° C. When complete, the mixture is concentrated and repeatedly distilled from ethanol (20 mL) to afford the title compound as an oil.

Preparation 21

(1R)-2-(Methylamino)-1-pyridin-2-ylethanol (2S)-2-(6Methoxy-2-naphthyl)propanoic Acid Salt.

2-(Methylamino)-1-pyridin-2-ylethanol (Preparation 20, Procedure B, approximately 1.16 g) is diluted with ethanol (15 mL) and (S)-Naproxen (1.75 g) is added. The mixture is heated to 75° C. and then cooled to 40° C. The mixture is further cooled to 0-5° C. The resulting slurry is stirred for at least 1 h, filtered and washed with cold ethanol (500 mL). The product is dried (vacuum oven, 50° C.) and then recrystallized from ethanol until the desired optical purity is obtained for the title compound. Physical characteristics. ¹H NMR (300 MHz, CDCl₃) δ 8.40, 8.05, 7.58, 7.40, 7.12, 7.02, 4.92, 3.87, 3.69, 3.00, 2.78, 2.23, 1.48; ¹³C NMR (75 MHz, CDCl₃) δ 181.6, 159.9, 157.2, 148.4, 138.8, 136.9, 133.2, 129.0, 128.9, 126.9, 126.6, 125.6, 122.5, 120.9, 118.5, 105.5, 69.0, 55.2, 55.0, 48.0, 33.1, 19.2. Anal. Found: C, 69.25; H. 6.89; N, 7.13.

Preparation 22.

(1R)-2-(Methylamino)-1-pyridin-2-ylethanol Dihydrochloride.

(1R)-2-(Methylamino)-1-pyridin-2-ylethanol (2S)-2-(6-methoxy-2-naphthyl)propanoic acid salt (Preparation 21, 6.1 g) is slurried in water (20 mL) and concentrated hydrochloric acid (4.25 mL) is added. The resulting slurry is heated to 50° C. for 3 h and is then cooled to 30° C. The slurry is filtered and the recovered Naproxen is washed with water (10 mL). The filtrate is concentrated to approximately 7 mL volume by vacuum distillation and diluted with ethanol (50 mL). The resulting solution is then concentrated to approximately 10 mL volume and cooled to 0° C. The mixture is filtered, washed with cold ethanol (10 mL) and dried (vacuum oven, 75° C.) to provide 3.4 g of the title compound. Physical characteristics. ¹H NMR (400 MHz, DMSO-d₆) δ 9.40, 8.79, 8.47, 8.04, 7.86, 5.42, 3.42, 3.23.

Preparation 23.

rac-2-(Methylamino)-1-(6-methylpyridin-2-yl)ethanol.

Potassium hydroxide (11.2 g) and water (0.45 mL) are added to acetonitrile (150 mL). Trimethylsulfonium iodide (20.4 g) and 6-methyl-2-pyridine carboxaldehyde (12.1 g) are then added. The reaction mixture is heated to 60° C. for 3 h. The reaction mixture is allowed to cool to room temperature. The precipitate is filtered, and the filtrate is concentrated in vacuo. The resulting crude epoxide (13.5 g) is dissolved in methanol (50 mL) and added to a 2.0 M solution of methylamine in methanol (250 mL). The reaction mixture is heated to reflux for 30 min. The reaction mixture is concentrated in vacuo. The resulting brown oil is purified by column chromatography (CHCl₃/methanol, 95/5, 90/10; CHCl₃/methanol/NH₄OH, 90/10/1). The resulting brown oil is suspended in hot methanol and the insoluble material filtered off. The filtrate is concentrated in vacuo to yield 3.657 g of the title compound as a yellow solid. Physical characteristics. M.p. 33-38° C.; ¹H NMR (400 MHz, DMSO-d₆) δ 7.64, 7.29, 7.10, 5.40, 4.63-4.60, 2.79-2.75, 2.61-2.54, 2.43, 2.30; MS (ESI+) m/z 167 (M+H)⁺.

Preparation 24.

rac-2-(Methylamino)-1-quinolin-2-ylethanol.

Potassium hydroxide (3.21 g) and water (0.13 mL) are added to acetonitrile (50 mL). Trimethylsulfonium iodide (5.84 g) and 2-quinoline carboxaldehyde (4.50 g) are then added. The reaction mixture is heated to 60° C. for 4 h. The reaction mixture is allowed to cool to room temperature and is diluted with Et₂O (25 mL). The precipitate is filtered off. The filtrate is concentrated in vacuo and the residue is re-subjected to the reaction conditions above and heated to 60° C. for 1 h. The reaction mixture is allowed to cool to room temperature and is diluted with Et₂O (25 mL). The precipitate is filtered off and the filtrate is concentrated in vacuo. The resulting crude epoxide (5.5 g) is dissolved in methanol (20 mL) and added to a 2.0 M solution of methylamine in methanol (100 mL). The reaction mixture is heated to reflux for 1 h. The reaction mixture is allowed to cool to room temperature and concentrated in vacuo. The resulting brown oil is purified by column chromatography (CHCl₃/methanol, 95/5, 90/10; CHCl₃/methanol/NH₄OH, 90/10/1) to yield 1.191 g of the title compound as a yellow-green oil. Physical characteristics. ¹H NMR (400 MHz, DMSO-d₆) δ 8.36-8.33, 7.98-7.94, 7.76-7.67, 7.59-7.54, 5.63, 4.88-4.84, 2.89-2.72, 2.32; MS (ESI+) m/z 203 (M+H)⁺.

Preparation 25.

2-Chloro-1-pyrimidin-2-ylethanone.

2-Acetylpyrimidine (7.37 g) and N,N-diisopropylethylamine (23.4 g) are dissolved in dry CH₂Cl₂ under nitrogen and cooled in an ice bath. Triisopropylsilyltriflate (17.9 ml) is added over 2-3 min and stirred over night. The solvent is evaporated and the residue treated with ether (200 ml), filtered and washed with sat. sodium bicarbonate solution (2×50 ml). Evaporation of the solvent gave a quantitative yield of 2-(1-((triisopropylsilyl)oxy)vinyl)pyridine as a red oil. Physical characteristics. ¹H NMR (300 MHz, CDCl₃) δ 1.15, 1.31, 4.90, 5.82, 7.16, 8.74; HRMS (FAB) nm/z 279.1898 (M+H)⁺.

N-Chlorosuccinimide (9.97 g) is added to a solution of the 2-(1-((triisopropylsilyl)oxy)vinyl)pyrimidine (17.3 g) in dry THF (120 mL) under nitrogen and the mixture is heated at 65° C. for 5 h. After cooling, ether (275 mL) is added and the solution is washed with sat. sodium bicarbonate solution (2×100 mL). The organic layer is dried (Na₂SO₄), filtered, and concentrated. The resulting oil is dissolved in hexane (250 mL), treated with MgSO₄, and filtered. Evaporation afforded 2-(2-chloro-1-((triisopropylsilyl)oxy)ethenyl))pyrimidine as a yellow oil in quantitative yield. Physical characteristics. ¹H NMR (300 MHz, CDCl₃) δ 1.13, 1.33, 6.97, 7.17, 8.68; HRMS (FAB) m/z 313.1509 (M+H)⁺.

2-(2-Chloro-1-((triisopropylsilyl)oxy)ethenyl))pyrimidine (19.4 g) is dissolved in acetonitrile (90 mL) and treated with 48% HF (10 mL) for 4 h. Sat. sodium bicarbonate solution (ca. 250 mL) is then added carefully to pH 7 and the mixture is extracted with CH₂Cl₂ (3×200 mL). After drying (Na₂SO₄), filtration and evaporation two oils are obtained. The upper colorless oil is decanted off and discarded while the lower oil crystallized to an oily solid. Chromatography over silica gel (500 g) eluting with 2.5% MeOH/CHCl₃ afforded 6.50 g of the title compound as a pale yellow solid. Physical characteristics. M.p. 73-80° C.; Anal. Found: C, 46.05; H, 3.09; N, 17.93.

Preparation 26.

2-Chloro-1-pyrimidin-2-ylethanol.

2-Chloro-1-pyrimidin-2-ylethanone (Preparation 25, 6.15 g) is dissolved in ethanol (125 mL) and CeCl₃.7H₂O (14.64 g) is added. After 10 min, sodium borohydride (1.49 g) is added over 2 min. After 1 h, the mixture is filtered and the filtrate evaporated. Sat. ammonium chloride solution (25 mL) is added followed by brine (250 mL) and the mixture adjusted to pH 3-4 with 1N HCl. The mixture is extracted with ethyl acetate (3×250 mL). The organic layer is concentrated. The resulting oil is chromatographed over silica gel (150 g) to give 3.85 g of the title compound as a pale yellow oil. Physical characteristics. ¹H NMR (300 MHz, CDCl₃) δ 4.09, 4.57, 5.17, 7.31, 8.80; Anal. Found: C, 45.08; H, 4.47; N, 17.46.

Preparation 27.

rac-2-(Methylamino)-1-pyrimidin-2-ylethanol.

2-Chloro-1-pyrimidin-2-ylethanol (Preparation 26, 3.525 g), sodium iodide (0.344 g) and a solution of methylamine (2 M in methanol, 160 ml) is placed in a pressure bottle. The bottle is sealed and heated at 62° C. for 17 h. The solvent is evaporated and the residue is stirred with 10% MeOH/CHCl₃. The mixture is filtered and the filtrate is concentrated. The resulting dark oil is chromatographed over silica gel (90 g) eluting with 5-10% MeOH/CH₂Cl₂ containing 1% triethylamine to afford 1.625 g of the title compound as an amber oil. Physical characteristics. ¹H NMR (400 MHz, CDCl₃) δ 2.53, 3.03, 3.21, 3.66, 5.03, 7.26, 8.77; HRMS (FAB) m/z 154.0979 (M+H)⁺.

Preparation 28.

Sodium pyrimidine-2-carboxylate.

To a slurry of 2-cyanopyrimidine (50 g) in water (100 ml) at 2° C. is added a solution of sodium hydroxide (50 wt %, 45.6 g) in water (30 ml) with an exotherm to 50° C. The mixture is stirred at 55° C. for 2 h, ethanol (500 ml) is added and the mixture concentrated in vacuo to an oil. Ethanol (250 ml) is added and the mixture concentrated to a paste. Ethanol (250 ml) is added and the mixture stirred at 15-20° C. for 30 min. The precipitate is collected by vacuum filtration, washed with ethanol (100 ml) and dried in a 75° C. vacuum oven to afford 67.57 g of the title compound as a white solid. Physical characteristics. ¹H NMR (400 MHz, CD₃OD) δ 7.53, 8.84; ¹³C NMR (100 MHz, CD₃OD) δ 123.7, 159.2, 163.6, 171.5.

Preparation 29.

N-Methoxy-N-methylpyrimidine-2-carboxamide.

Sodium pyrimidine-2-carboxylate (Preparation 28, 154.05 g), imidazole hydrochloride (119.3 g), and 1,1-carbonyldiimidazole (195 g) is slurried in acetonitrile (700 nA). The mixture is warmed from 15° C. to 52° C. over 0.5 h. Carbon dioxide is vigorously evolved between 30 and 50° C. The mixture is stirred 1 h at 52° C. then cooled to 15° C. and N,O-dimethylhydroxylamine hydrochloride (131.90 g) is added with an exotherm to 34° C. The mixture is cooled to 14° C. and methylene chloride (300 ml) and water (500 ml) is added. The pH is adjusted from 7.6 to 1.6 with aqueous sulfuric acid (6.13 M, 226 ml). The phases are separated and the lower aqueous phase washed with methylene chloride (500 ml). To the combined organics is added water (300 ml) and the pH adjusted to 1.18 with aqueous sulfuric acid (6.13 M, 5.1 ml). The phases are separated and the organics washed with saturated aq. sodium bicarbonate (300 ml). All three aqueous phases are serial back extracted with methylene chloride (500 ml). The bicarbonate wash is back extracted with methylene chloride (500 nml). The combined organics are dried (MgSO₄) and concentrated in vacuo to a thin slurry. The residue is slurried in methylene chloride (200 ml) and the solids filtered off. The filtrate is concentrated to afford 160.7 g of the title compound as an oil. Physical characteristics. ¹H NMR (400 MHz, CDCl₃) δ 3.38, 3.69, 7.39, 8.82; ¹³C NMR (100 MHz, CDCl₃) δ 32.05, 61.62, 121.34, 157.

Preparation 30.

4-(Pyrimidin-2-ylcarbonyl)morpholine.

Following the general procedure of Preparation 29 and making non-critical variations, but substituting morpholine for N,O-dimethylhydroxylamine hydrochloride the title compound is obtained. Physical characteristics. ¹H NMR (400 MHz, CDCl₃) δ 3.40, 3.69, 3.83, 7.38, 8.83; ¹³C NMR (100 MHz, CDCl₃) δ 42.2, 47.1, 66.6, 66.7, 121.2, 157.4, 161.8, 165.0.

Preparation 31.

S-Phenyl Pyrimidine-2-carbothioate.

Sodium pyrimidine-2-carboxylate (Preparation 28, 5.06 g), imidazole hydrochloride (4.23 g), and 1,1-carbonyldiimidazole (7.14 g) is slurried in acetonitrile (40 mL). The mixture is warmed to 52° C. and stirred for 1 h. The mixture is cooled to 7° C. and thiophenol (4.52 ml, 44.0 mmol, 1.27 eq) is added. The mixture is stirred at 17° C. for 10 minutes then poured into water (25 ml). Toluene (25 ml) is added and the phases separated. The aqueous layer is extracted with toluene (2×25 ml). The combined organic layers are dried (MgSO₄) and then concentrated to an oil. Branched octanes (42 g) is added, the mixture seeded and the resultant slurry cooled to 0° C. The precipitate is collected by vacuum filtration, washed with branched octanes and dried in a nitrogen stream to give a solid. The solid is partitioned between toluene (44 g) and water (25 ml) at approximately 30° C. The phases are separated and the aqueous layer is extracted with toluene (3×25 ml). The combined organic layers are dried (MgSO₄) and then concentrated to 45 g net weight. Branched octanes (35 g) is added, the mixture seeded and the precipitate is collected by vacuum filtration, washed with branched octanes and dried in a nitrogen stream to afford 5.75 g of the title compound as a solid. Physical characteristics. ¹H NMR (400 MHz, CDCl₃) δ 7.47, 7.54, 8.97; ¹³C NMR (100 MHz, CDCl₃) δ 123.6, 127.5, 129.3, 129.6, 134.6, 157.8, 159.1.

Preparation 32.

tert-Butyl (1-(pyrimidin-2-yl)ethanon-2-yl)(methyl)carbamate.

Procedure A. To a solution of tert-butyl dimethylcarbamate (57.8 g) in N,N,N′,N′-tetramethylethylenediamine (70 ml) and MTBE (485 g) is added sec-butyl lithium (1.4 M in cyclohexane, 300 ml) over 0.5 h while maintaining the temperature below −65° C. The mixture is stirred at −65° C. for 0.5 h. then magnesium bromide diethyl etherate (111.07 g) is added with an exotherm to −60° C. The resultant slurry is allowed to warm to −11° C. over 0.5 h then cooled to −72° C. The slurry is cannulated into a −72° C. solution of N-methoxy-N-methylpyrimidine-2-carboxamide (Preparation 29, 27.2 g) in methylene chloride (400 ml) with an exotherm to −60° C. and rinsed in with MTBE (25 ml). The mixture is warmed to 0° C. over 45 min then cooled to −27° C. Acetone (30.5 ml) is added. The mixture is cooled to −29° C., then a solution of acetic acid (63.7 g) in water (303 ml) is added with an exotherm to 11° C. The mixture is warmed to 20° C. and the phases separated. The organic layer is washed with saturated aq. sodium bicarbonate (250 ml) and the aqueous phases serial back extracted with MTBE (350 ml). The combined organics are dried (MgSO₄) and concentrated in vacuo to a net weight of 85 g. Toluene (200 ml) is added and the mixture concentrated to 128 g net weight. Branched octanes (205 g) is added to the cloud point, the mixture seeded and the product allowed to precipitate for 15 minutes with stirring. The slurry is cooled to −19° C. and the precipitate collected by vacuum filtration, washed with branched octanes (82 g) and dried in a nitrogen stream to afford 29.27 g of the title compound as a solid. Physical characteristics. ¹H NMR (400 MHz, CDCl₃) δ 1.38, 1.49, 3.00, 4.83, 4.92, 7.50, 8.94; ¹³C NMR (100 MHz, CDCl₃) δ 28.11, 28.30, 35.57, 35.71, 56.11, 56.61, 79.96, 123.25, 123.36, 157.56, 157.65.

Procedure B. Following the general procedure of Preparation 32, Procedure A and making non-critical variations, but substituting 4-(pyrimidin-2-ylcarbonyl)morpholine (Preparation 30) or S-phenyl pyrimidine-2-carbothioate (Preparation 31) for N-methoxy-N-methylpyrimidine-2-carboxamide the title compound is obtained.

Preparation 33.

Tert-Butyl (2R)-2-hydroxy-2-pyrimidin-2-ylethyl(methyl)carbamate.

In a glove box, triethylamine (6.6 g) is added carefully with stifling to formic acid (4.6 g) in a glass vial and stirring is continued until the mixture had cooled to room temperature. A 50 mL Schlenk flask is charged with [(η⁶C₆H)RuCl₂]₂ (200 mg), (R)(R)-TsDPEN (350 mg), anhydrous i-PrOH (10 mL), and triethylamine (0.35 mL). The Schlenk flask is removed from the glove box and placed on a Schlenk line, a reflux condenser attached, and the reaction mixture is heated to 75° C. for 1 h under nitrogen. The reaction is then cooled to 0° C. giving a solid which is collected by filtration. The solid is washed with diethyl ether and air-dried giving 228 mg of (η⁶C₆H₆)Ru[(R,R)-TsDPEN]Cl. To a 50 mL RB flask in a glove box is added (η⁶C₆H))Ru[(R,R)-TsDPEN]Cl (17 mg) followed by the mixture of the triethylamine/formic acid solution prepared above. The mixture is allowed to stir at room temperature for 20 min and tert-butyl 2-oxo-2-pyrimidin-2-ylethyl(methyl)carbanate (Preparation 32, 1.33 g) is added. The mixture is stirred at room temperature for 17 h, poured into water (75 mL), and extracted with EtOAc (3×100 mL). The combined organic layers are washed with 1 M aq. NaHCO₃ (50 mL) and brine (50 mL). The organic layer is dried (MgSO₄), filtered, and concentrated to afford 1.17 g of the title compound as an oil. Physical characteristics. ¹H NMR (CDCl₃) 8.66, 7.20, 4.95, 3.69, 3.45, 288, 1.30. MS m/z 276 (MNa⁺).

Preparation 34.

(1R)-2-(Methylamino)-1-pyrimidin-2-ylethanol Dihydrochloride.

A 6 N solution of aq. HCl (5 mL) is added to tert-butyl (2R)-2-hydroxy-2-pyrimidin-2-ylethyl(methyl)carbamate (Preparation 33, 1.17 g) at room temperature. After 2.5 h, reaction mixture is concentrated in vacuo using 3×10 mL portions of ethanol to assist in water removal. The oil is dissolved in ethanol, heated to ca. 50° C. and THF is added until slightly turbid at this temperature. The solution is allowed to cool to room temperature. The resulting solid is collected by filtration and washed with ethanol/THF (50/50) followed by diethyl ether to afford 0.78 g of the title compound. Physical properties. ¹HNM (D₂O) 8.85, 7.62, 5.17, 3.45, 3.30, 2.63; ¹³C NMR (D₂O) 165.1, 158.3, 122.3, 68.4, 52.5, 33.6.

Preparation 35.

2-Chloroacetylpyrazine.

2-Acetylpyrazine (53.9 g) is dissolved in CH₂Cl₂ (700 mL) and N,N-diisopropylethylamine (231 mL) is added. The mixture is cooled in an ice-water bath and TIPSOTf (130.4 mL) is added over 1.5 hours. The mixture is allowed to warm to room temperature overnight and then is concentrated. The residue is suspended in ether (1.2 L) and is washed with saturated aq. NaHCO₃ (2×700 mL). The organic layer is separated, dried (MgSO₄), filtered and concentrated in vacuo to furnish 132.9 g of 2-(1-(triisopropylsilyloxy)ethenyl)pyrazine as a brown oil.

NCS (64.78 g) is added to a solution of 2-[1-(triisopropylsilyloxy)ethenyl]pyrazine (132.9 g) in THF (640 mL). The mixture is heated to reflux for 3 h and then allowed to cool to room temperature. The mixture is diluted with ether (1.5 L), and is washed with saturated aq. NaHCO₃ (2×700 mL). The organic layer is separated, dried (MgSO₄), filtered and concentrated in vacuo to afford 169.45 g of 2-(2-chloro-1-(triisopropylsilyloxy)ethenyl]pyrazine as a brown oil.

2-(2-Chloro-1-(triisopropylsilyloxy)ethenyl)pyrazine (169.45 g) is dissolved in acetonitrile (470 mL) and 48% aqueous HF (73.54 mL) is added. After 10 h, the pH of the solution is carefully adjusted to ca. 8 with saturated aq. NaHCO₃. The mixture is diluted with CH₂Cl₂ (1.5 L). The organic layer is separated and the aqueous layer is extracted with CH₂Cl₂ (2×1.0 L). The combined organic layers are dried (MgSO₄), filtered and concentration in vacuo. The crude product is purified by silica gel column chromatography eluting with CH₂Cl₂ to afford 60.1 g of the title compound as a light yellow solid. Physical characteristics. M.p. 82.6-83.8° C. (dec.); ¹H NMR (300 MHz, CDCl₃) δ 9.23, 8.80, 8.64, 5.01; ¹³C NMR (75 MHz, CDCl₃) δ 191.4, 148.4, 145.6, 143.5, 143.3, 46.4; MS (EI) m/z 156 (M+).

Preparation 36.

rac-2-Chloro-1-pyrazin-2-ylethanol.

Sodium borohydride (0.285 g) is added to a cold (0° C.) solution of 2-chloroacetyl-pyrazine (Preparation 35, 1.075 g) and cerium chloride heptahydrate (2.81 g) in methanol (18 mL). The mixture is allowed to warm to room temperature and after 1 h is quenched with water. The aqueous layer is extracted with diethyl ether. The combined organic layers are dried (MgSO₄), filtered and concentrated in vacuo. The crude product is purified by chromatography (3/1 diethyl ether/hexane) to afford 0.607 g of the title compound as a light yellow liquid. Physical characteristics. ¹H NMR (400 MHz, CDCl₃) δ 8.80, 8.56, 5.07, 3.93, 3.87; ¹³C NMR (100 MHz, CDCl₃) δ 154.2, 144.2, 143.5, 143.3, 71.7, 48.9; HRMS (FAB) m/z 159.0325 (M+H)⁺.

Preparation 37.

rac-2-(Methylamino)-1-pyrazin-2-ylethanol.

A mixture of rac-2-chloro-1-pyrazin-2-ylethanol (11.8 g), NaI (1.12 g), and a solution of methylamine (2.0 M in methanol, 370 mL) is heated to 60° C. for 20 h. The mixture is cooled to room temperature and concentrated in vacuo. The crude product is purified by column chromatography (CH₂Cl₂/methanol) to afford the title compound as a light orange oil. Physical characteristics. ¹H NMR (400 MHz, DMSO-d₆) δ 8.78, 8.58, 5.75, 4.92, 3.06, 2.94, 2.44; ¹³C NMR (100 MHz, DMSO-d₆) δ 157.1, 143.3, 143.2, 142.8, 69.4, 55.4, 34.7; MS (CI) m/z 154 (M+H)⁺.

Preparation 38.

2-Bromo-1-pyrazin-2-ylethanone Hydrobromide.

A 1 L round-bottomed flask was charged with 2-acetylpyrazine (25 g), glacial acetic acid (175 mL), and a 30 wt % solution of HBr in acetic acid (40 mL). Pyridinium tribromide (70 g) was added to the mixture all at once as a solid. The slurry was allowed to stir for 1 h at room temperature. This resulting solution was poured into diethyl ether (1.5 L) giving a yellow solid which was recovered by gravity filtration, washed with CH₃CN (3×500 mL) and then diethyl ether (2×250 nL) to afford 34.9 g of the title compound. Physical characteristics. MS m/z 201, 202.

Preparation 39.

Tert-Butyl (1-(pyrazin-2-yl)ethanon-2-yl)(methyl)carbamate.

A 2 L round-bottomed flask was charged with 2-bromo-1-pyrazin-2-ylethanone hydrobromide (Preparation 38, 49.2 g) followed by THF (1 L). The resulting slurry was cooled to ca. 0-5° C. (ice bath). To this solution was added a 2 M solution of methylamine in THF (350 mL) giving rise to an exotherm with the solution reaching 15° C. After 20 minutes, (Boc)₂O (75 g) was added all at once as a solid at 5° C. The reaction mixture was allowed to stir for 30 min and then additional (Boc)₂O (10 g) was added and the reaction mixture allowed to stir for an additional 30 min at 5° C. The reaction mixture was allowed to warm to room temperature and was then filtered through a short pad of silica gel using ethyl acetate to wash the silica gel. The filtrate was concentrated in vacuo. The resulting oil was purified by column chromatography (hexane/EtOAc, 9/1; 4/1) to afford 25.5 g of the title compound. Physical characteristics. ¹H NMR (CD₃CN) δ 9.10, 8.78, 8.64, 4.78, 2.89, 2.86, 1.41, 1.28; MS m/z 274 (MNa⁺)

Preparation 40.

Tert-Butyl (2R)-2-hydroxy-2-pyrazin-2-ylethyl(methyl)carbamate.

A 50 mL Schlenk flask was charged with [(η⁶C₆H₆)RuCl₂]₂ (200 mg), (R)(R)-TsDPEN (350 mg), anhydrous i-PrOH (10 mL), and triethylamine (0.35 mL). The Schlenk flask was removed from the glove box and placed on a Schlenk line, a reflux condenser attached, and the reaction mixture was heated to 75° C. for 1 h under nitrogen. The reaction was then cooled to 0° C. giving a solid which was collected by filtration. The solid was washed with diethyl ether and air-dried giving 228 mg of (η⁶C₆H₆)Ru[(R,R)-TsDPEN]Cl. A solution of formic acid and triethylamine was prepared by adding triethylamine (91 g) to formic acid (65 g) cooled in an ice bath under a nitrogen atmosphere. The ice bath was removed and to this solution was added (η⁶C₆H(O)Ru[(R,R)-TsDPEN]Cl (106 mg) and the solution allowed to stir at room temperature for 30 min. To this solution was added tert-butyl (1-(pyrazin-2-yl)ethanon-2-yl)(methyl)carbanate (Preparation 39, 27.98 g) and the mixture was stirred at room temperature for 21 h. Additional (η6C₆H₆)Ru[(R,R)-TsDPEN]Cl (110 mg) was added and the mixture stirred at room temperature for an additional 24 h. The mixture was poured into water (500 mL) and ethyl acetate (500 mL). The aqueous layer was extracted with ethyl acetate (500 mL). The combined ethyl acetate layers were extracted with 1 N aq. NaHCO₃ (2×250 mL), water (250 mL), and brine (250 mL). The ethyl acetate layer was then dried (MgSO₄), filtered, and concentrated to give 27.5 g of the title compound as a light brown oil. Physical characteristics. ¹H NMR (CD₃CN) δ 8.66, 8.46, 4.91, 3.55, 3.4, 2.80, 1.34, 1.25; MS m/z 276 (MNa⁺).

Preparation 41.

(1R)-2-(Methylamino)-1-pyrazin-2-ylethanol.

A mixture of tert-butyl (2R)-2-hydroxy-2-pyrazin-2-ylethyl(methyl)carbanate (Preparation 40, 27.5 g) and 6 N aq. HCl (105 mL) was stirred at room temperature for 20 min. The mixture was concentrated in vacuo using ethanol to azeotrope residual water. To the residue was added 20% aq. NaOH until the pH reached 11. This aqueous solution was extracted with EtOAc (2×250 mL). The pH was then adjusted to 14 and NaCl was added to saturate the aqueous layer. This was then extracted with EtOAc (2×200 mL). The combined EtOAc layers were dried (MgSO₄), filtered, and concentrated to afford a solid. The aqueous layer was further extracted with CH₃CN (2×250 mL). The combined organic layers were dried (MgSO₄), filtered, and concentrated to afford a solid. The combined solids were dissolved in hot MTBE, filtered, and allowed to cool first to room temperature and then to 5° C. in the refrigerator giving 8.4 g of the title compound as yellow crystals. Physical characteristics. ¹H NMR (DMSO-d₆) δ 8.72, 8.51, 4.74, 2.81, 2.72, 2.26; ¹³C NMR (DMSO-d₆) δ 159.3, 144.0, 143.8, 143.5, 71.7, 58.2, 36.7; MS m/z 154 (MH⁺).

Preparation 42.

3-(1-((Triisopropylsilyl)oxy)vinyl)pyridazine.

Triisopropylsilyl triflate (26.4 g) is added over 4 min. to an ice cooled solution of 3-acetylpyridazine (9.57 g) and diisopropylethylamine (30.4 g) in dry CH₂Cl₂ (100 mL) under nitrogen. After 4 h, the solvent is evaporated and the residue extracted with diethyl ether (150 mL). The organic layer is washed with saturated aq. sodium bicarbonate solution (2×60 mL) followed by brine (60 mL), dried (MgSO₄), filtered and evaporated. The crude product is purified by column chromatography (hexanes/EtOAc, 9/1) to afford 13.4 g of the title compound as a yellow oil. Physical characteristics. ¹H NMR (400 MHz, CDCl₃) δ 9.10, 7.85, 7.52, 5.98, 4.74, 1.35, 1.16; HRMS (ESI) m/z 279.1879.

Preparation 43.

3-((E)-2-Chloro-1-((triisopropylsilyl)oxy)vinyl)pyridazine.

N-Chlorosuccinimide (9.7 g) is added to a solution of 3-(1-((triisopropylsilyl)oxy)vinyl)pyridazine (Preparation 42, 13.4 g) in dry THF (110 mL) under nitrogen. The mixture is heated at 65° C. for 20 h and then allowed to cool. The mixture is diluted with diethyl ether (500 mL) and washed with saturated aq. sodium bicarbonate solution (2×100 mL) followed by brine (200 mL). The organic layer is dried (Na₂SO₄), filtered and concentrated. The crude product is purified by column chromatography (hexanes/EtOAc, 9/1; 4/1) to afford 7.66 g of the title compound as a yellow oil. Physical characteristics. ¹H NMR (400 MHz, CDCl₃) δ 9.15, 7.71, 7.53, 6.83, 1.33, 1.12; HUMS (ESI) m/z 313.1496.

Preparation 44.

2-Chloro-1-pyridazin-3-ylethanone.

A 48% solution of HF (4 mL) is added to a solution of 3-((E)-2-chloro-1-((triisopropylsilyl)oxy)vinyl)pyridazine (Preparation 43, 7.9 g) in acetonitrile (35 mL). The reaction mixture is stirred for 4 h and then is neutralized with a saturated sodium bicarbonate solution. The mixture is extracted with CH₂Cl₂ (3×100 mL). The combined organic layers are dried (MgSO₄), filtered and concentrated. The crude product is recrystallized from acetone/hexane to afford the title compound as a yellow solid. Physical characteristics. M.p. 104-106° C.; ¹H NMR (400 MHz, DMSO-d₆) δ 9.37, 8.19, 7.96, 5.46, HRMS (ESI) m/z 157.0168.

Preparation 45.

rac-2-Chloro-1-pyridazin-3-ylethanol.

Cerium(III) chloride heptahydrate (7.95 g) followed by sodium borohydride (0.905 g) are added to a stirred solution of 2-chloro-1-pyridazin-3-ylethanone (Preparation 44, 3.34 g) in ethanol (75 mL). After 4 h, the reaction mixture is filtered and concentrated in vacuo. The residue is treated with ice (50 g) and the pH is adjusted to 3-4. The mixture is extracted with chloroform (100 mL). The organic layer is dried (MgSO₄), filtration and concentrated. The crude product is purified by column chromatography (chloroform/methanol, 99/1-97/3) to afford 2.12 g of the title compound as an amber gum. Physical characteristics. ¹H NMR (400 MHz, CDCl₃) δ 9.19, 7.77, 7.58, 5.27, 4.07, 3.95; HRMS (ESI) m/z 159.0324.

Preparation 46.

rac-2-(Methylamino)-1-pyridazin-3-ylethanol.

A mixture of rac-2-chloro-1-pyridazin-3-ylethanol (Preparation 45, 2.08 g), sodium iodide (0.211 g) and a solution of methylamine (100 mL, 2 M in methanol) is sealed in a pressure bottle and heated in an oil bath at 67° C. for 18 h. After cooling, the solvent is evaporated and the residue is triturated with CH₂Cl₂ (50 mL). The resulting solid is filtered and dissolved in methanol (50 mL). The solution is treated with polystyrene resin bound 1,5,7-triazabicyclo(4,4,0)dec-2-ene cross linked with 2% DVB (Fluka). After 3 days, the resin is filtered and the filtrate concentrated. The residue is suspended in CH₂Cl₂ (20 mL) and filtered. The filtrate is concentrated to afford 0.933 g of the title compound as a cream solid. Physical characteristics. M.p. 76-77° C.; ¹H NMR (400 MHz, CDCl₃) δ 9.13, 7.74, 7.52, 5.06, 3.16, 2.94, 2.51; HRMS (ESI) m/z 154.0977.

### Example 1

rac-N-(4-Chlorobenzyl)-2-(((2-hydroxy-2-pyridin-3-ylethyl)(methyl)amino)methyl)-7-methyl-4-oxo-4,7-dihydrothieno[2,3-b]pyridine-5-carboxamide.

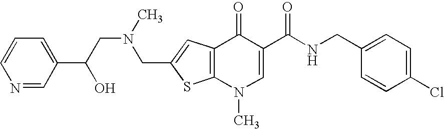

N-(4-Chlorobenzyl)-2-(chloromethyl)-7-methyl-4-oxo-4,7-dihydrothieno[2,3-b]pyridine-5-carboxamide (Preparation 1, 0.250 g) is suspended in DMF (14 mL), and N,N-diisopropylethylamine (0.46 mL) and 2-(methylamino)-1-pyridin-3-ylethanol hydrobromide (Preparation 18, 0.305 g) are added. The reaction mixture is heated to 90° C. for 2 h. The reaction mixture is allowed to cool to room temperature and is poured into water (50 mL). The resulting off-white solid is filtered and purified by column chromatography (CH₂Cl₂/methanol; 98/2, 97/3). The resulting pale yellow solid is recrystallized from methanol to yield 0.083 g of the title compound as a pale yellow solid. Physical characteristics. M.p. 160-162° C.; ¹H NMR (400 MHz, DMSO-d₆) δ 10.60, 8.69, 8.53-8.52, 8.47-8.45, 7.73-7.70, 7.41-7.30, 5.39, 4.83-4.79, 4.55, 3.90, 3.85, 2.72-2.59, 2.32; ¹³C NMR (100 MHz, CDCl₃) δ 171.2, 163.7, 149.8, 147.5, 147.4, 144.5, 139.1, 138.0, 130.7, 129.8, 128.4, 127.7, 122.5, 113.6, 47.9, 42.1, 41.8; MS (ESI+) m/z 497 (M+H)⁺; HRMS (FAB) m/z 497.1407 (M+H)⁺. Anal. Found: C, 59.06; H, 5.35; N, 10.84; Cl, 6.85; S, 6.20

### Example 2

(+)-N-(4-Chlorobenzyl)-2-(((2-hydroxy-2-pyridin-3-ylethyl)(methyl)amino)methyl)-7-methyl-4-oxo-4,7-dihydrothieno[2,3-b]pyridine-5-carboxamide.

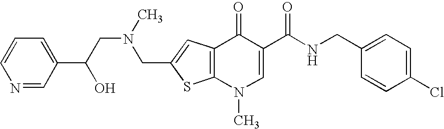

rac-N-(4-Chlorobenzyl)-2-(((2-hydroxy-2-pyridin-3-ylethyl)(methyl)amino)methyl)-7-methyl-4-oxo-4,7-dihydrothieno[2,3-b]pyridine-5-carboxamide (Example 1) is resolved preparatively on a 5×50 cm Chiralcel OD-H column (Chiral Technologies), at a column temperature of 30° C. The mobile phase is 50% ethanol/50% heptane (v/v) with a flow rate of 84 mL/min. Peaks are detected by UV at 230 nm. A 447 mg sample is injected. The more slowly eluting enantiomer is isolated and then further purified by recrystallization from methanol then ethyl acetate to yield 0.153 g of the title compound as an off-white solid. Physical characteristics. M.p. 132-135° C.; ¹H NMR (400 MHz, DMSO-d₆) δ 10.60, 8.69, 8.53, 8.47-8.45, 7.73-7.70, 7.41-7.33, 7.29, 5.39, 4.84-4.79, 4.55, 3.90, 3.84, 2.72-2.59, 2.31; ¹³C NMR (100 MHz, CDCl₃) δ 172.3, 164.7, 150.8, 148.5, 148.4, 145.5, 140.1, 139.8, 139.0, 134.3, 131.7, 130.8, 129.5, 128.7, 123.5, 120.5, 114.6, 68.9, 64.3, 56.7, 43.1, 42.8, 41.7; MS (ESI+) m/z 497 (M+H)⁺, HRMS (FAB) m/z 497.1414 (M+H)⁺; [α]²⁵_{D}=+41 (c 0.76, methylene chloride). Anal. Found: C, 60.24; H, 5.12; N, 11.12; Cl, 7.04; S, 6.33.

### Example 3

rac-N-(4-Chlorobenzyl)-2-(((2-hydroxy-2-pyridin-4-ylethyl)(methyl)amino)methyl)-7-methyl-4-oxo-4,7-dihydrothieno[2,3-b]pyridine-5-carboxamide.

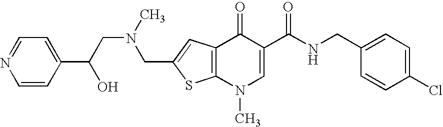

N-(4-Chlorobenzyl)-2-(chloromethyl)-7-methyl-4-oxo-4,7-dihydrothieno[2,3-b]pyridine-5-carboxamide (Preparation 1, 0.500 g) is suspended in DMF (30 mL), and N,N-diisopropylethylamine (0.46 mL) and 2-(methylamino)-1-pyridin-4-ylethanol (Preparation 19, 0.399 g) are added. The reaction mixture is heated to 90° C. for 2 h. The reaction mixture is allowed to cool to room temperature and is poured into water (100 mL). The resulting off-white solid is filtered and purified by column chromatography (CH₂Cl₂methanol; 99/1, 98/2, 97/3). The resulting pale yellow solid is recrystallized from methanol to yield 0.402 g of the title compound as a pale yellow solid. Physical characteristics. M.p. 185-188° C.; ¹H NMR (400 MHz, DMSO-d₆) δ 10.60, 8.69, 8.50-8.48, 7.41-7.30, 5.48, 4.80-4.76, 4.55, 3.90; ¹³C NMR (100 MHz, CDCl₃) δ 173.0, 164.8, 150.8, 150.6, 149.7, 144.8, 137.3, 136.5, 132.8, 131.5, 128.9, 128.7, 122.5, 120.8, 115.9, 68.4, 64.2, 57.2, 43.2, 42.6, 41.8; MS (ESI+) m/z 497 (M+H)⁺; HRMS (FAB) m/z 497.1413 (M+H)⁺. Anal. Found: C, 59.90; H, 5.14; N, 11.13; Cl, 7.08; S, 6.37

### Example 4

rac-N-(4-Chlorobenzyl)-2-(((2-hydroxy-2-pyridin-2-ylethyl)(methyl)amino)methyl)-7-methyl-4-oxo-4,7-dihydrothieno[2,3-b]pyridine-5-carboxamide.

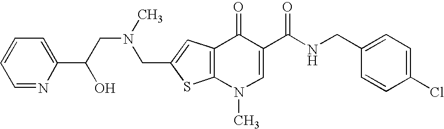

N-(4-Chlorobenzyl)-2-(chloromethyl)-7-methyl-4-oxo-4,7-dihydrothieno[2,3-b]pyridine-5-carboxamide (Preparation 1, 0.500 g) is suspended in DMF (30 mL), and N,N-diisopropylethylamine (0.46 mL) and 2-(methylamino)-1-pyridin-2-ylethanol (Preparation 20, 0.399 g) are added. The reaction mixture is heated to 90° C. for 2 h. The reaction mixture is allowed to cool to room temperature and is poured into water (100 mL). The resulting off-white solid is filtered and purified by column chromatography (CH₂Cl₂/methanol; 99/1, 98/2, 97/3). The resulting pale yellow solid is recrystallized from methanol to yield 0.290 g of the title compound as a pale yellow solid. Physical characteristics. M.p. 145-147° C.; ¹H NMR (400 MHz, DMSO-d₆) δ 10.61, 8.69, 8.47-8.46, 7.80-7.76, 7.50-7.48, 7.41-7.33, 7.30-7.24, 5.38, 4.83-4.79, 4.55, 3.92, 3.89-3.81, 2.84-2.80, 2.73-2.68, 2.32; ¹³C NMR (100 MHz, CDCl₃) δ 173.0, 164.9, 160.6, 150.6, 148.7, 144.7, 137.3, 136.8, 132.8, 131.5, 128.9, 128.6, 122.6, 120.5, 115.8, 70.3, 63.5, 57.2, 43.1, 42.6, 42.0; MS (ESI+) m/z 497 (M+H)⁺; HRMS (FAB) m/z 497.1428 (M+H)⁺. Anal. Found: C, 59.67; H, 5.10; N, 11.09; Cl, 7.12; S, 6.36.

### Example 5

(+)-N-(4-Chlorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide.

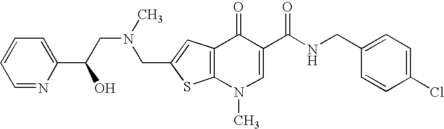

Procedure A. (1R)-2-(Methylamino)-1-pyridin-2-ylethanol (Preparation 22, 0.228 g) is dissolved in DMF (23 mL). N,N-diisopropylethylamine (0.26 mL) and N-(4-chlorobenzyl)-2-(chloromethyl)-7-methyl-4-oxo-4,7-dihydrothieno[2,3-b]pyridine-5-carboxamide (Preparation 1, 0.381 g) are added, and the reaction mixture is heated to 90° C. for 2 h. The reaction mixture is allowed to cool to room temperature and is poured into water (60 mL). The resulting tan solid is filtered and purified by column chromatography (CH₂Cl₂/methanol, 99/1; 98:2; 97/3) to yield 0.337 g of a pale yellow solid which is recrystallized from acetonitrile to yield 0.208 g of the title compound as a white solid. Physical characteristics. M.p. 139-144° C.; ¹H NMR (400 MHz, DMSO-d₆) δ 10.61, 8.69, 8.48-8.46, 7.79-7.76, 7.50-7.48, 7.41-7.24, 5.38, 4.83-4.79, 4.55, 3.92, 3.89-3.81, 2.86-2.80, 2.73-2.68, 2.32; ¹³C NMR (100 MHz, CDCl₃) δ 173.0, 164.9, 160.6, 150.6, 148.7, 144.7, 137.3, 136.8, 132.8, 131.5, 128.9, 128.7, 122.6, 120.6, 115.8, 70.3, 63.5, 57.2, 43.2, 42.6, 42.1; MS (ESI+) m/z 497 (M+H)⁺; [α]²⁵_{D}=+38 (c 0.92, methylene chloride); Anal. Found: C, 60.03; H, 5.12; N, 11.17; Cl, 7.19; S, 6.46.

Procedure B. Powdered potassium carbonate (2.63 g) is added to a suspension of N-(4-chlorobenzyl)-2-(chloromethyl)-7-methyl-4-oxo-4,7-dihydrothieno[2,3-b]pyridine-5-carboxamide (Preparation 1, 2.25 g) and (1R)-2-(methylamino)-1-pyridin-2-ylethanol dihydrochloride (Preparation 22, 1.40 g) in acetonitrile (40 mL). The mixture is heated to 75-80° C. for 12-18 hours and then filtered through a pad of Celite at 60-80° C. The cake is washed with acetonitrile (2×2 mL). The combined filtrates are concentrated in vacuo to a volume of approximately 25 mL. The resulting slurry is cooled to 0-5° C., stirred for 30 min, and filtered. The solid is washed with acetonitrile (2×3 mL) and dried in a vacuum oven at 60° C. for 18 h to provide 2.70 g of the title compound.

### Example 6

rac-N-(4-Chlorobenzyl)-2-(((2-hydroxy-2-(6-methylpyridin-2-yl)ethyl)(methyl)amino)methyl)-7-methyl-4-oxo-4,7-dihydrothieno[2,3-b]pyridine-5-carboxamide.

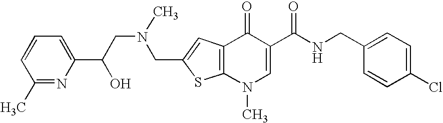

2-(Methylamino)-1-(6-methylpyridin-2-yl)ethanol (Preparation 23, 0.327 g) is dissolved in DMF (30 mL), and N,N-diisopropylethylamine (0.34 mL) and N-(4-chlorobenzyl)-2-(chloromethyl)-7-methyl-4-oxo-4,7-dihydrothieno[2,3-b]pyridine-5-carboxamide (Preparation 1, 0.500 g) are added. The reaction mixture is heated to 90° C. for 1 h. The reaction mixture is allowed to cool to room temperature and is poured into water (100 mL) and extracted with CH₂Cl₂ (4×50 mL). The combined organic layers are dried (MgSO₄), filtered, and concentrated in vacuo. The resulting off-white solid is purified by column chromatography (CH₂Cl₂/methanol; 99/1, 98/2). The resulting pale yellow solid is recrystallized from ethyl acetate to yield 0.464 g of the title compounds as an off-white solid. Physical characteristics. M.p. 158-162° C.; ¹H NMR (400 MHz, DMSO-d₆) δ 10.62, 8.70, 7.67-7.63, 7.41-7.27, 7.11-7.09, 5.29, 4.77-4.73, 4.55, 3.92, 3.90-3.81, 2.82-2.78, 2.71-2.66, 2.41, 2.32; ¹³C NMR (100 MHz, CDCl₃) δ 173.0, 165.0, 160.0, 157.4, 150.5, 144.6, 138.5, 137.4, 137.0, 132.7, 131.5, 128.9, 128.6, 122.1, 117.4, 115.7, 70.5, 63.8, 57.2, 43.1, 42.5, 42.2, 24.4; MS (ESI+) m/z 511 (M+H)⁺. Anal. Found: C, 60.82; H, 5.28; N, 10.80; Cl, 6.61; S, 5.99.

### Example 7

rac-N-(4-Chlorobenzyl)-2-(((2-hydroxy-2-quinolin-2-ylethyl)(methyl)amino)methyl)-7-methyl-4-oxo-4,7-dihydrothieno[2,3-b]pyridine-5-carboxamide.

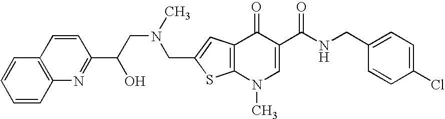

2-(Methylamino)-1-quinolin-2-ylethanol (Preparation 24, 0.398 g) is dissolved in DMF (30 μL), and N,N-diisopropylethylamine (0.34 μL) and N-(4-chlorobenzyl)-2-(chloromethyl)-7-methyl-4-oxo-4,7-dihydrothieno[2,3-b]pyridine-5-carboxamide (Preparation 1, 0.500 g) are added. The reaction mixture is heated to 90° C. for 1 h. The reaction mixture is allowed to cool to room temperature and is poured into water (60 mL) and extracted with CH₂Cl₂ (4×50 μL). The combined organic layers are dried (MgSO₄), filtered, and concentrated in vacuo. The resulting yellow-green solid is purified by column chromatography (CH₂Cl₂/methanol; 99/1, 98/2). The resulting pale yellow solid is recrystallized from acetonitrile then methanol to yield 0.363 g of the title compound as a white solid. Physical characteristics. M.p. 155-160° C.; ¹H NMR (400 MHz, DMSO-d₆) δ 10.60, 8.64, 8.35-8.33, 7.97-7.93, 7.73-7.69, 7.66-7.64, 7.59-7.55, 7.41-7.32, 7.28, 5.61, 4.99-4.95, 4.55, 3.85, 3.67, 2.94-2.81, 2.34; MS (ESI+) m/z 547 (M+H)⁺. Anal. Found: C, 63.41; H, 5.00; N, 10.13; Cl, 6.51; S, 5.88.

### Example 8

rac-N-(4-Chlorobenzyl)-2-(((2-hydroxy-2-pyrimidin-2-ylethyl)(methyl)amino)methyl)-7-methyl-4-oxo-4,7-dihydrothieno[2,3-b]pyridine-5-carboxamide.

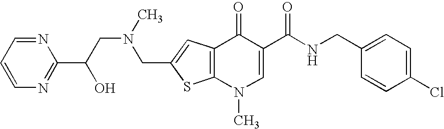

2-(Methylamino)-1-pyrimidin-2-ylethanol (Preparation 27, 0.206 g), N-(4-chlorobenzyl)-2-(chloromethyl)-7-methyl-4-oxo-4,7-dihydrothieno[2,3-b]pyridine-5-carboxamide (Preparation 1, 0.345 g), and N,N-diisopropylethylamine (0.235 mL) are shaken in dry DMF (10 mL) under nitrogen for 4 days. The DMF is evaporated (40° C./1 Torr) and the residue is dissolved in CHCl₃ (75 ml). The mixture is washed with water (2×50 mL), brine (25 mL) and dried (MgSO₄). The organic layer is filtered and concentrated to afford 0.41 g of the title compound as a white solid. Physical characteristics. M.p. 181° C.; ¹H NMR (300 MHz, CDCl₃) δ 2.48, 2.95, 3.10, 3.96, 3.91, 4.18, 4.64, 5.05, 7.29, 7.41, 8.61, 8.77; HRMS (FAB) m/z 498.1395 (M+H)⁺. Anal. Found: C, 57.37; H, 4.89; N, 13.80.

### Example 9

N-(4-Chlorobenzyl)-2-((((2R)-2-hydroxy-2-pyrimidin-2-ylethyl(methyl)amino)methyl)-7-methyl-4-oxo-4,7-dihydrothieno[2,3-b]pyridine-5-carboxamide.

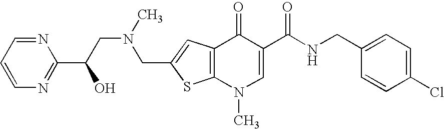

To a 5 L 3-necked round bottom flask fitted with mechanical stirring, thermocouple, reflux condenser and nitrogen inlet is charged (1R)-2-(methylamino)-1-pyrimidin-2-ylethanol dihydrochloride (Preparation 34, 40.5 g), N-(4-chlorobenzyl)-2-(chloromethyl)-7-methyl-4-oxo-4,7-dihydrothieno[2,3-b]pyridine-5-carboxamide (Preparation 1, 56.8 g), powdered potassium carbonate (97.0 g), sodium iodide (0.22 g), acetonitrile (2 L), and water (3 mL). The solution is degassed by three vacuum nitrogen purges and then heated under reflux (˜75° C.) for 16 h. The slurry is filtered at 75-80° C. and the cake washed with 75° C. acetonitrile (500 mL). The combined filtrates are concentrated to ˜500 mL volume by vacuum distillation and cooled to −5° C. The product is collected by filtration, washed with −5° C. acetonitrile (200 mL) and dried in a vacuum oven at 65° C. for 8 hours. The crude product is dissolved in CH₂Cl₂ (550 ml) and filtered through a 0.6 micron filter. The resulting solution is concentrated to 200 mL volume using atmospheric distillation and acetonitrile (500 mL) is added. The resulting solution is concentrated to 200 mL volume and 250 mL of acetonitrile is added. The resulting mixture is concentrated with atmospheric distillation to ˜20 mL volume and cooled to −10° C. for 30 min. The resulting slurry is filtered and the product washed with cold acetonitrile (2×100 mL). The cake was dried in a vacuum oven at 70° C. for 48 h. to afford 68 g of the title compound. Physical characteristics. ¹H NMR (400 MHz, CDCl₃) δ 10.62, 8.75, 8.59, 7.38, 7.31-7.23, 5.00, 4.62, 3.9, 3.88, 3.86, 3.04-2.88, 2.44; ¹³C NMR (100 MHz, CDCl₃) δ 172.9, 169.4, 165.0, 157.0, 144.4, 138.9, 137.3, 132.7, 131.5, 128.9, 128.6, 121.2, 119.7, 115.6, 84.2, 71.8, 62.5, 57.4, 43.0, 42.5, 42.4; [α]⁵D=+37° (CHCl₃, c=1). Anal. Found: C, 57.88, H, 4.89, N, 13.97; Cl, 7.09; S, 6.40.

Preparation 47.

N-(4-Chlorobenzyl)-7-methyl-2-((methyl(2-oxo-2-(pyrazin-2-yl)ethyl)amino)methyl)-4-oxo-4,7-dihydrothieno[2,3-b]pyridine-5-carboxamide. Cesium carbonate (390 mg) and 3 Å molecular sieves are added to a solution of 2-chloroacetylpyrazine (Preparation 35, 204 mg) and N-(4-chlorobenzyl)-7-methyl-2-((methylamino)methyl)-4-oxo-4,7-dihydrothieno[2,3-b]pyridine-5-carboxamide (Preparation 3, 374 mg) in DMF (5.0 mL). The reaction mixture is heated to 60° C. for 17 h. The mixture is concentrated and the residue is dissolved in chloroform. The solution is washed with water and the organic layer is concentrated. The crude product is purified by chromatography over silica gel with 5% CH₃OH in CH₂Cl₂ to afford 245 mg of the title compound as a white solid. Physical characteristics. MS (CI) m/z 496 (M+H)⁺.

### Example 10

rac-N-(4-Chlorobenzyl)-2-(((2-hydroxy-2-pyrazin-2-ylethyl)(methyl)amino)methyl)-7-methyl-4-oxo-4,7-dihydrothieno[2,3-b]pyridine-5-carboxamide.

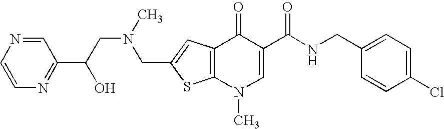

Sodium borohydride (19 mg) is added to a suspension of N-(4-chlorobenzyl)-7-methyl-2-((methyl(2-oxo-2-(pyrazin-2-yl)ethyl)amino)methyl)-4-oxo-4,7-dihydrothieno[2,3-b]pyridine-5-carboxamide (Preparation 47, 100 mg) in MeOH (10.0 mL). The solvent is evaporated and the residue is chromatographed over silica gel with 5% MeOH in CH₂Cl₂ to afford 26 mg of the title compound as white crystals. Physical characteristics. ¹H NMR (400 MHz, DMSO-d₆) δ 10.52, 8.72, 8.69, 8.55, 7.39, 7.30, 7.28, 5.63, 4.89, 4.53, 3.89, 3.81, 2.80, 2.72, 1.98; HRMS (FAB) m/z 498.1360 (M+H)⁺.

### Example 11

N-(4-Chlorobenzyl)-2-((((2R)-2-hydroxy-2-pyrazin-2-ylethyl)(methyl)amino)methyl)-7-methyl-4-oxo-4,7-dihydrothieno[2,3-b]pyridine-5-carboxamide.

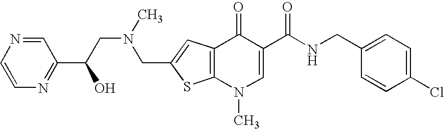

N-(4-Chlorobenzyl)-2-(chloromethyl)-7-methyl-4-oxo-4,7-dihydrothieno[2,3-b]-pyridine-5-carboxamide (Preparation 1, 381 mg) and (1R)-2-(methylamino)-1-pyrazin-2-ylethanol (Preparation 41, 100 mg) are dissolved in DNIF (3 mL). Diisopropylethylamine (226 μL) and 3 Å sieves (100 mg) are added. The reaction mixture is placed on a shaker block at 60° C. for 72 h. The solvent is evaporated and the residue is dissolved into CH₂Cl₂. The organic layer is washed with water, dried (MgSO₄), treatment with decolorizing carbon, filtered, and concentrated. The crude product is purified by column chromatography (CHCl₂C, methanol, 95/5) followed by recrystallization from EtOAc/diethyl ether to afford 310 nig of the title compound as white crystals. Physical characteristics. ¹H NMR (400 MHz, DMSO-d₆) δ 10.60, 8.72, 8.69, 8.55, 7.41-7.28, 5.65, 4.88, 4.54, 3.97, 3.89, 3.81, 3.77, 3.34, 2.88, 2.86, 2.50, 2.32; MS (CI) m/z 498 (M+H)⁺. Anal. Found: C, 57.69; H, 4.89; N, 13.68.

### Example 12

N-(4-Chlorobenzyl)-2-(((2-hydroxy-2-pyridazin-3-ylethyl)(methyl amino)methyl)-7-methyl-4-oxo-4,7-dihydrothieno[2,3-b]pyridine-5-carboxamide.

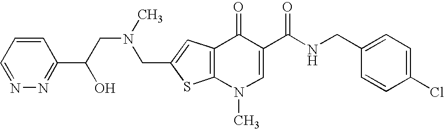

A mixture of rac-2-(methylamino)-1-pyridazin-3-ylethanol (Preparation 46, 0.200 g), N-(4-chlorobenzyl)-2-(chloromethyl)-7-methyl-4-oxo-4,7-diydrothieno[2,3-b]-pyridine-5-carboxamide (Preparation 1, 0.375 g), and diisopropylethylamine (0.181 g) in dry DMF (7 mL) is heated at 55° C. for 5 h. The reaction mixture is concentrated and the residue is dissolved in chloroform (100 mL). The mixture is washed with water (2×20 mL) and brine (20 mL). The organic layer is dried (MgSO₄), filtered and concentrated. The residue is purified by column chromatography (chloroform/methanol, 98/2-94/6) to afford 0.366 g of the title compound as a white solid. Physical characteristics. M.p. 153-155° C.; ¹H NMR (400 MHz, DMSO-d₆) δ 10.58, 9.13, 8.69, 7.70, 7.39, 7.34, 7.28, 5.72, 5.06, 4.54, 3.90, 3.83, 2.87, 2.79, 2.33. Anal. Found: C, 57.78. H, 4.94; N, 13.90.

### Example 13

rac-N-(4-Chlorobenzyl)-7-ethyl-2-(((2-hydroxy-2-pyrazin-2-ylethyl)(methyl)amino)methyl)-4-oxo-4,7-dihydrothieno[2,3-b]pyridine-5-carboxamide.

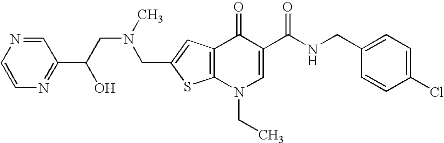

N,N-Diisopropylethylamine (220 μL) is added to a solution of N-(4-chlorobenzyl)-2-(chloromethyl)-7-ethyl-4-oxo-4,7-dihydrothieno[2,3-b]pyridine-5-carboxamide (Preparation 11, 250 mg) and rac-2-(methylamino)-1-pyrazin-2-ylethanol (Preparation 37, 193 mg) in DMF (14 mL). The mixture is stirred at 90° C. for 1 h and then allowed to cool to room temperature. The mixture is concentrated in vacuo and triturated with ethyl acetate to afford 206 mg of the title compound as a white solid. Physical characteristics. M.p. 130-133° C.; ¹H NMR (300 MHz, DMSO-d₆) δ 10.58, 8.72, 8.55, 7.36, 7.27, 5.64, 4.88, 4.54, 4.23, 3.81, 2.83, 2.32, 1.42; HRMS (FAB) m/z 512.1501 (M+H)⁺.

### Example 14

rac-N-(4-Chlorobenzyl)-7-ethyl-2-(((2-hydroxy-2-pyrimidin-2-ylethyl)(methylamino)methyl)-4-oxo-4,7-dihydrothieno[2,3-b]pyridine-5-carboxamide.

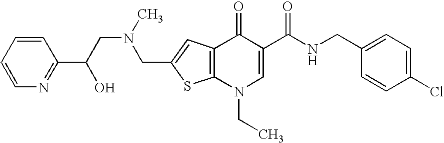

A mixture of N-(4-chlorobenzyl)-2-(chloromethyl)-7-ethyl-4-oxo-4,7-dihydrothieno[2,3-b]pyridine-5-carboxamide (Preparation 11, 0.3 g), 2-(methylamino)-1-pyridin-2-ylethanol hydrochloride (Preparation 20, 0.3 g), and N,N-diisopropyl-ethylamine (0.4 mL) in DMF (17 mL) is stirred at 90° C. for 4 h. The reaction mixture is cooled to room temperature and concentrated in vacuo. The crude product is purified by column chromatography (CH₂Cl₂/methanol, 98/2) and triturated with diethyl ether to afford 0.24 g of the title compound as an off-white powder. Physical characteristics. ¹H NMR (300 MHz, DMSO-d₆) δ 10.60, 8.72, 8.47, 7.77, 7.49, 7.43-7.22, 5.38, 4.81, 4.54, 4.27, 3.85, 2.87-2.64, 2.32, 1.43; MS (ESI+) m/z 502 (M+H). Anal. Found: C, 57.20. H, 4.97; N, 8.30; Cl, 7.01; S, 12.54.

### Example 15

rac-N-(4-Chlorobenzyl)-7-propyl-2-(((2-hydroxy-2-pyridin-2-ylethyl)(methyl)amino)methyl)-4-oxo-4,7-dihydrothieno[2,3-b]pyridine-5-carboxamide.

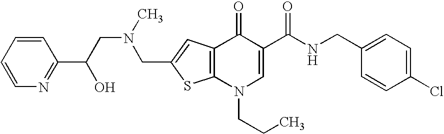

A mixture of N-(4-chlorobenzyl)-2-(chloromethyl)-7-propyl-4-oxo-4,7-dihydrothieno[2,3-b]pyridine-5-carboxamide (Preparation 13, 0.3 g), rac-2-(methylamino)-1-pyridin-2-ylethanol hydrochloride (Preparation 20, 0.3 g), and N,N-diisopropylethylamine (0.4 mL) in DMF (17 mL) is stirred at 90° C. for 4 h. The reaction mixture is cooled to room temperature and concentrated in vacuo. The crude product is purified by column chromatography (CH₂Cl₂/methanol, 98/2) and triturated with diethyl ether to afford 0.13 g of the title compound as a white powder. Physical characteristics. ¹H NMR (300 MHz, DMSO-d₆) δ 10.60, 8.70, 8.46, 7.77, 7.49, 7.43-7.32, 7.30-7.21, 5.38, 4.81, 4.54, 4.21, 4.15-4.07, 3.84, 2.92-2.63, 1.85, 0.91. Anal. Found: C, 61.52; H, 5.63; N, 10.40; Cl, 6.77; S, 6.14.

### Example 16

rac-N-(4-Chlorobenzyl)-2-(((2-hydroxy-2-pyrazin-2-ylethyl)(methyl)amino)methyl)-4-oxo-7-propyl-4,7-dihydrothieno[2,3-b]pyridine-5-carboxamide.

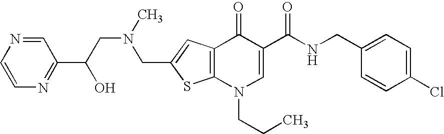

A mixture of N-(4-chlorobenzyl)-2-(chloromethyl)-7-propyl-4-oxo-4,7-dihydrothieno[2,3-b]pyridine-5-carboxamide (Preparation 13, 0.25 g), rac-2-(methylamino)-2-pyrazin-2-ylethanol (Preparation 37, 0.19 g), and N,N-diisopropylethylamine (0.21 mL) in DMF (14 mL) is stirred at 90° C. for 1 h. The reaction mixture is cooled to room temperature and concentrated in vacuo. The crude product is purified by column chromatography (CH₂Cl₂/methanol, 98/2) and triturated with methanol to afford 0.11 g of the title compound as an off-white powder. Physical characteristics. ¹H NMR (300 MHz, DMSO-d₃) δ 10.59, 8.71, 8.54, 7.43-7.31, 7.26, 5.65, 4.88, 4.54, 4.17, 3.81, 2.94-2.71, 2.31, 1.84, 0.90. Anal. Found: C, 59.08. H, 5.38. N, 13.11; Cl, 6.75; S, 6.09.

Preparation 48.

N-(4-Chlorobenzyl)-7-((2,2-dimethyl-1,3-dioxolan-4-yl)methyl)-2-((((2R)-2-hydroxy-2-pyridin-2-ylethyl)(methyl)amino)methyl)-4-oxo-4,7-dihydrothieno[2,3-b]pyridine-5-carboxamide.

Cesium carbonate (260 mg) and 3 Å molecular sieves (100 mg) are added to a solution of 2-(chloromethyl)-N-((4-chlorophenyl)methyl)-7-((2,2-dimethyl-1,3-dioxolan-4-yl)methyl)-4,7-dihydro-4-oxothieno[2,3-b]pyridine-5-carboxamide (Preparation 4, 336 mg) and (R)-2-(methylamino)-1-pyridin-2-ylethanol (Preparation 22, 100 mg) in DMF (3.0 mL). The reaction mixture is placed on a shaker block at 60° C. for 5 h and then the solvent is evaporated. The residue is dissolve in 10% MeOH in CH₂Cl₂ and washed with water. The organic layer is dried (MgSO₄), filtered, concentrated. The crude product is purified by chromatograph over silica gel with 5% MeOH in CH₂Cl₂ to afford 244 mg of the title compound as a white solid. Physical characteristics. ¹H NMR (400 MHz, DMSO-d₆) δ 10.52, 8.68, 8.46, 7.77, 7.48, 7.39, 7.34, 7.28, 7.25, 5.34, 4.80, 4.53, 4.45, 4.27, 4.14, 3.85, 3.77, 2.82, 2.70, 2.32, 1.33, 1.23; HRMS (FAB) m/z 597.1945 (M+H)⁺. Anal Found: C, 60.03; H, 5.69; N, 9.02.

### Example 17

N-(4-Chlorobenzyl)-7-(2,3-dihydroxypropyl)-2-((((2R)-2-hydroxy-2-pyridin-2-ylethyl)(methyl)amino)methyl)-4-oxo-4,7-dihydrothieno[2,3-b]pyridine-5-carboxamide.

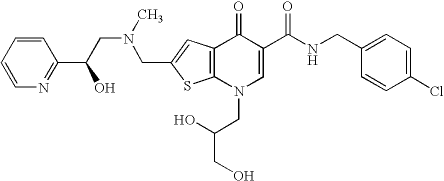

N-(4-Chlorobenzyl)-7-((2,2-dimethyl-1,3-dioxolan-4-yl)methyl)-2-((((2R)-2-hydroxy-2-pyridin-2-ylethyl)(methyl)amino)methyl)-4-oxo-4,7-dihydrothieno[2,3-b]pyridine-5-carboxamide (Preparation 48, 150 mg) is dissolved in THF (5 mL) and 65% perchloric acid (0.2 mL) is added. The reaction mixture is stirred for 5 h at 35° C. and is then poured into sat. NaHCO₃ solution. The mixture is extracted with EtOAc (100 mL). The organic layer is dried (MgSO₄), filtered and concentrated. The residue is purified by chromatography over silica gel with 5% MeOH in CH₂Cl₂ followed by trituration with EtOAc to afford 75 mg of the title compound as a white solid. Physical characteristics. ¹H NMR (400 MHz, DMSO-d₆) δ 10.45, 8.60, 8.47, 7.77, 7.48, 7.39, 7.34, 7.28, 7.24, 5.36, 5.31, 4.99, 4.80, 4.53, 4.29, 4.12, 3.85, 3.50, 3.39, 2.80, 2.71, 2.31; MS (EI) m/z 556 (M+); HRMS (FAB) m/z 557.1625 (M+H)⁺.

Preparation 49.

N-(4-Chlorobenzyl)-2-((((2R)-2-hydroxy-2-pyridin-2-ylethyl)(methyl)amino)methyl)-4-oxo-7-(3-(tetrahydro-2H-pyran-2-yloxy)propyl)-4,7-dihydrothieno[2,3-b]pyridine-5-carboxamide.

Cesium carbonate (260 mg) and 3 Å molecular sieves (100 mg) are added to a solution of N-(4-chlorobenzyl)-2-(chloromethyl)-4-oxo-7-(3-(tetrahydro-2H-pyran-2-yloxy)propyl)-4,7-dihydrothieno[2,3-b]pyridine-5-carboxamide (Preparation 6, 300 mg) and (R)-2-(methylamino)-1-pyridin-2-ylethanol (Preparation 22, 100 mg) in DMF (2.5 mL). The reaction mixture is placed on a shaker block at 60° C. for 17 h and then the solvent is evaporated. The residue is purified by chromatography over silica gel with 5% MeOH in CH₂Cl₂ to afford 154 mg of the title compound as a white solid. Physical characteristics. ¹H NMR (400 MHz, DMSO-d₆) δ 10.52, 8.71, 8.68, 8.61, 8.46, 7.91, 7.77, 7.48, 7.39, 7.33, 7.29, 7.25, 5.79, 5.57, 5.37, 4.80, 4.70, 4.53, 4.48, 4.38, 4.32, 3.85, 3.68, 3.38, 2.79, 2.32, 2.09, 1.63, 1.54, 1.39; HRMS (FAB) m/z 625.2278 (M+H)⁺.

### Example 18

N-(4-Chlorobenzyl)-7-(3-hydroxypropyl)-2-((((2R)-2-hydroxy-2-pyridin-2-ylethyl)(methyl)amino)methyl)-4-oxo-4,7-dihydrothieno[2,3-b]pyridine-5-carboxamide.

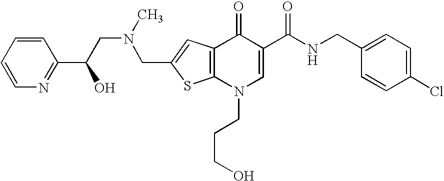

N-(4-Chlorobenzyl)-2-((((2R)-2-hydroxy-2-pyridin-2-pyridin-2-ylethyl)(methyl)amino)methyl)-4-oxo-7-(3-(tetrahydro-2H-pyran-2-yloxy)propyl)-4,7-dihydrothieno[2,3-b]pyridine-5-carboxamide (Preparation 49, 118 mg) is dissolved in THF (5 mL) and a solution of 65% perchloric acid (0.2 mL) in water (0.2 mL) is added. The reaction is stirred at 60° C. for 5 h and is then poured into sat. NaHCO₃ solution. The mixture is extracted with EtOAc (100 mL). The organic layer is dried (MgSO₄), filtered and concentrated. The residue is chromatographed over silica gel with 5% MeOH in CH₂Cl₂ to afford 97 mg of the title compound. Physical characteristics. ¹H NMR (400 MHz, DMSO-d₆) δ 10.56, 8.68, 8.47, 7.77, 7.48, 7.39, 7.34, 7.29, 7.24, 5.37, 4.81, 4.76, 4.70, 4.54, 4.37, 4.29, 3.84, 3.45, 3.33, 2.81, 2.71, 1.98; MS (EI) m/z 540 (M+); HRMS (FAB) m/z 541.1677 (M+H)⁺.

Preparation 50.

N-(4-Chlorobenzyl)-2-(((2-hydroxy-2-pyrimidin-2-ylethyl)(methyl)amino)methyl)-4-oxo-7-(3-(tetrahydro-2H-pyran-2-yloxypropyl)-4,7-dihydrothieno[2,3-b]pyridine-5-carboxamide.

Diisopropylethylamine (0.175 mL) and 3 Å molecular sieves (100 mg) are added to a solution of (4-chlorobenzyl)-2-(chloromethyl)-4-oxo-7-(3-(tetrahydro-2H-pyran-2-yloxy)propyl)-4,7-dihydrothieno[2,3-b]pyridine-5-carboxamide (Preparation 6, 255 mg) and rac-2-(methylamino)-1-pyrimidin-2-ylethanol (Preparation 27, 150 mg) in DMF (3 mL). The reaction mixture is placed on a shaker block at 60° C. for 17 h and then the solvent is evaporated. The residue is purified by column chromatography (CH₂Cl₂/methanol, 9515) to afford the title compound. Physical characteristics. ¹H NMR (400 MHz, DMSO-d₆) δ 10.58, 8.78, 8.68, 7.42, 7.38, 7.34, 7.26, 5.34, 4.85, 4.53, 4.48, 4.30, 3.77, 3.68, 3.38, 2.99, 2.77, 2.50, 2.28, 2.07, 1.63, 1.54, 1.38; MS (CI) m/z 626 (MHE; HRMS (ESI) m/z 626.2209 (M+H)⁺.

### Example 19

rac-N-(4-Chlorobenzyl)-7-(3-hydroxypropyl)-2-(((2-hydroxy-2-pyrimidin-2-ylethyl)(methyl)amino)methyl)-4-oxo-4,7-dihydrothieno[2,3-b]pyridine-5-carboxamide.

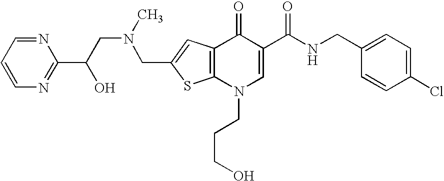

N-(4-Chlorobenzyl)-2-(((2-hydroxy-2-pyrimidin-2-ylethyl)(methyl)amino]methyl}-4-oxo-7-(3-(tetrahydro-2H-pyran-2-yloxy)propyl)-4,7-dihydrothieno[2,3-b]pyridine-5-carboxamide (Preparation 50, 140 mg) is dissolved in THF (5 mL) and a solution of 65% perchloric acid (0.2 mL) in water (0.1 mL) is added. The reaction is stirred at room temperature for 3 h and is then poured into sat. NaHCO₃ solution. The mixture is extracted with EtOAc (150 mL). The organic layer is dried (MgSO₄), filtered and concentrated. The residue is purified by column chromatography (CH₂Cl₂/methanol, 95/5) to afford 68 mg of the title compound. Physical characteristics. ¹H NMR (400 MHz, DMSO-d₆) δ 10.60, 8.78, 8.67, 7.43-7.35, 7.25, 5.35, 4.81, 4.79, 4.53, 4.25, 3.77, 3.45, 3.34, 3.32, 2.92, 2.79, 2.50, 2.28, 1.95, MS (CI) m/z 542 (M+H)⁺, HRMS (ESI) m/z 542.1617 (M+H)⁺.

### Example 20

N-(4-Chlorobenzyl)-7-(2-hydroxyethyl)-2-((((2R)-2-hydroxy-2-pyridin-2-ylethyl)(methyl)amino)methyl)-4-oxo-4,7-dihydrothieno[2,3-b]pyridine-5-carboxamide.

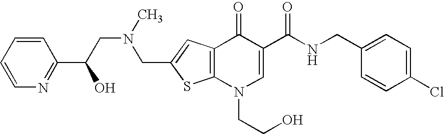

Cesium carbonate (260 mg) and 3 Å sieves (100 mg) are added to a solution of N-(4-chlorobenzyl)-2-(chloromethyl)-7-(2-hydroxyethyl)]-4-oxo-4,7-dihydrothieno[2,3-b]-pyridine-5-carboxamide (Preparation 9, 270 mg) and (1R)-2-(methylamino)-1-pyridin-2-ylethanol (Preparation 22, 100 mg) in DMF (3.0 mL). The reaction mixture is placed on a shaker block at 60° C. for 2 h and then the solvent is evaporated. The residue is purified by column chromatography (CH₂Cl₂/methanol, 95/5) and then by recrystallization from EtOAc to afford 147 mg of the title compound as a white solid. Physical characteristics. ¹H NMR (400 MHz, DMSO-d₆) δ 10.53, 8.62, 8.46, 7.77, 7.48, 7.39, 7.33, 7.28, 7.25, 5.35, 5.15, 4.80, 4.54, 4.26, 3.85, 3.78, 2.81, 2.70, 2.32; HRMS (FAB) m/z 527.1520 (M+H)⁺.

### Example 21

rac-N-(4-Chlorobenzyl)-2-(((2-hydroxy-2-pyrazin-2-ylethyl)(methyl)amino)methyl)-7-(2-methoxyethyl)-4-oxo-4,7-dihydrothieno[2,3-b]pyridine-5-carboxamide.

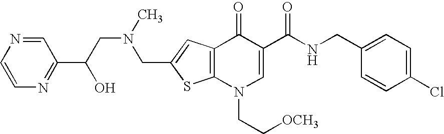

A mixture of N-(4-chlorobenzyl)-2-(chloromethyl)-7-(2-methoxyethyl)-4-oxo-4,7-dihydrothieno[2,3-b]pyridine-5-carboxamide (Preparation 15, 0.25 g), rac-2-(methylamino)-2-pyrazin-2-ylethanol (Preparation 37, 0.19 g), and N,N-diisopropylethylamine (0.21 mL) in DMF (14 mL) is stirred at 90° C. for 1 h. The reaction mixture is cooled to room temperature and concentrated in vacuo. The crude product is purified by column chromatography (CH₂Cl₂/methanol, 100/1; 50/1; 33/1; 20/1) and crystallized from EtOAc/hexanes at −10° C. to afford 0.118 g of the title compound as a tan solid. Physical characteristics. M.p. 122-123; ¹H NM (400 MHz, DMSO-d₆) δ 10.57, 8.73, 8.63, 8.55, 7.41-7.33, 7.27, 5.63, 4.88, 4.54, 4.38, 3.84, 3.80, 3.73, 3.26, 2.88, 2.78, 2.32; ¹³C NMR (100 MHz, DMSO-d₆) δ 172.3, 164.6, 158.8, 150.1, 145.3, 143.7, 143.6, 139.8, 139.0, 131.7, 131.3, 129.5, 128.7, 120.2, 114.5, 71.1, 69.1, 62.7, 58.7, 56.7, 56.2, 42.9, 41.8; MS (ESI+) m/z 542 (M+H)⁺. Anal. Found: C, 57.46; H, 5.31; N, 12.82; Cl, 6.49; S, 5.91.

Preparation 51.

N-(4-Chlorobenzyl)-2-(((2R)-2-hydroxy-2-pyrazin-2-ylethyl)(methylamino)methyl)-4-oxo-7-(2-(2-(tetrahydro-2H-pyran-2-yloxy)ethoxy)ethyl)-4,7-dihydrothieno[2,3-b]pyridine-5-carboxamide.

Diisopropylethylamine (96 μL) and 3 Å sieves are added to a solution of N-(4-chlorobenzyl)-2-(chloromethyl)-4-oxo-7-(2-(2-(tetrahydro-2H-pyran-2-yloxy)ethoxy)ethyl)-4,7-dihydrothieno[2,3-b]pyridine-5-carboxamide (Preparation 17, 250 mg) and (1R)-2-(methylamino)-1-pyrazin-2-ylethanol (Preparation 41, 80 mg) in DMF (3 mL). The mixture is heated on a shaker block at 60° C. for 4 h. The solvent is evaporated and the residue is purified by column chromatography (CH₂Cl₂/methanol, 95/5) to afford 187 mg of the title compound as a yellow oil. Physical characteristics. ¹H NMR (400 MHz, DMSO-d₆) δ 10.57, 8.73, 8.64, 8.55, 7.40, 7.38, 7.35, 7.26, 5.65, 5.64, 4.88, 4.53, 4.40, 3.82, 3.59, 3.55, 3.52, 3.36, 3.34, 3.24, 2.89, 2.85, 2.78, 2.50, 2.31, 1.48, 1.42, 1.31; MS (CI) m/z 656 (M+H)⁺; HRMS (ESI) 656.2313 (M+H)⁺.

### Example 22

N-(4-Chlorobenzyl)-2-((((2R)-2-hydroxy-2-pyrazin-2-ylethyl)(methyl)amino)methyl)-4-oxo-7-(2-(2-(tetrahydro-2H-pyran-2-yloxy)ethoxy)ethyl-4,7-dihydrothieno[2,3-b]pyridine-5-carboxamide.

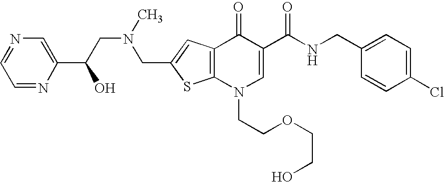

N-(4-Chlorobenzyl)-2-(((2R)-2-hydroxy-2-pyrazin-2-ylethyl)(methyl)amino)methyl)-4-oxo-7-(2-(2-(tetrahydro-2H-pyran-2-yloxy)ethoxy)ethyl)-4,7-dihydrothieno[2,3-b]pyridine-5-carboxamide (Preparation 51, 80 mg) is dissolved in THF (5 mL) and a solution of 65% perchloric acid (10 drops) is added. The reaction is stirred at room temperature for 3 h and is then diluted with EtOAc. The mixture is washed with saturated aq. NaHCO₃ solution. The organic layer is dried (MgSO₄), filtered and concentrated. The crude product is purified by column chromatography (CH₂Cl₂/methanol, 95/5) to afford 39 mg of the title compound. Physical characteristics. ¹H NMR (400 MHz, DMSO-d₆) δ 10.58, 8.73, 8.65, 8.55, 7.41, 7.39, 7.35, 7.26, 5.66, 4.88, 4.61, 4.55, 4.53, 4.37, 3.84, 3.44, 3.34, 2.87, 2.80, 2.50, 2.31, 1.23; MS (FAB) W/z 572 (M+H); HRMS (FAB) m/z 572.1729 (M+H)⁺.

Preparation 52.

Ethyl 4-Hydroxy-2-(hydroxymethyl)thieno[2,3-b]pyridine-5-carboxylate.

Ethyl 4-hydroxythieno[2,3-b]pyridine-5-carboxylate (3.0 kg, prepared as described in U.S. Pat. No. 6,239,142) is dissolved in THF (150 L) and cooled to −70° C. Freshly prepared LDA (2.9 eq.) is added while maintaining the temperature at −70° C. DMF (3.0 eq.) is added, and the reaction is stirred at −70° C. for 1.5 h. The reaction mixture is quenched into 10% KH₂PO₄. Solvents are removed by distillation and remaining water is removed azeotropic distillation with ethanol. The resulting crude ethyl 2-formyl-4-hydroxythieno[2,3-b]pyridine-5-carboxylate is dissolved in CH₂Cl₂/methanol (50/50, 60 L). The solution is cooled to −10° C. and NaBH₄ (250 g) is added. The reaction mixture is stirred at room temperature for 3 h, quenched with water (40 L), and adjusted to pH 3-5 with 10% HC (15 L). The mixture is extracted with CH₂Cl₂ (2×30 L). The combined organic layers are distilled and the crude product is triturated with hot ethyl acetate. The resulting solid is dissolved in hot THF/toluene (50/50) and purified by column chromatography (EtOAc/toluene, 50/50; EtOAc) to yield 2.04 kg of the title compound as an off-white solid. Physical characteristics. ¹H NMR (400 MHz, DMSO-d₆) δ 8.67, 7.31, 5.75, 4.73, 4.36, 1.35.

Preparation 53.

Ethyl 2-(hydroxymethyl)-7-methyl-4-oxo-4,7-dihydrothieno[2,3-b]pyridine-5-carboxylate.

Ethyl 4-hydroxy-2-(hydroxymethyl)thieno[2,3-b]pyridine-5-carboxylate (Preparation 52, 10.0 g) is suspended in DMF (300 mL). Potassium carbonate (8.20 g) and iodomethane (2.95 mL) are added. The reaction mixture is stirred at room temperature for 4 h. Additional iodomethane (1.23 mL) is added and stirring is continued for 1 h. The reaction mixture is filtered, and the filtrate is concentrated in vacuo. The resulting off-white solid is purified by column chromatography (CH₂Cl₂/methanol, 98/2; 95/5; 90/10) to yield 8.45 g of the title compound as a white solid. Physical characteristics. ¹H NMR (400 MHz, DMSO-d₆) δ 8.49, 7.23, 5.76, 4.68, 4.22, 1.27; MS (ESI+) m/z 268 (M+H)⁺.

Preparation 54.

Ethyl 2-(chloromethyl)-7-methyl-4-oxo-4,7-dihydrothieno[2,3-b]pyridine-5-carboxylate.

Ethyl 2-(hydroxymethyl)-7-methyl-4-oxo-4,7-dihydrothieno[1,2-b]pyridine-5-carboxylate (Preparation 53, 7.50 g) is suspended in CH₂Cl₂ (300 mL). N,N-diisopropylethylamine (7.4 mL) is added and the reaction mixture is cooled to 0° C. Methanesulfonyl chloride (3.3 mL) is added and the reaction mixture is stirred at room temperature for 18 h. A saturated aq. sodium bicarbonate solution (200 mL) and CH₂Cl, (250 mL) are added. The mixture is stirred for 30 minutes. The organic layer is removed and the aqueous layer extracted with CH₂Cl₂ (250 mL). The combined organic layers are dried (MgSO₄), filtered, and concentrated in vacuo. The resulting solid is triturated with EtOAc to yield 5.68 g of the title compound as a tan solid. Physical characteristics. ¹H NMR (400 MHz, DMSO-d₆) δ 8.53, 7.50, 5.14, 4.22, 1.27.

Preparation 55.

Ethyl 2-((((2R)-2-Hydroxy-2-pyridin-2-ylethyl)(methyl)aminomethyl)-7-methyl-4-oxo-4,7-dihydrothieno[2,3-b]pyridine-5-carboxylate.

(1R)-2-(Methylamino)-1-pyridin-2-ylethanol dihydrochloride (Preparation 22, 4.73 g) is suspended in chloroform (300 mL). N,N-Diisopropylethylamine (12.2 mL) is added followed by ethyl 2-(chloromethyl)-7-methyl-4-oxo-4,7-dihydrothieno[2,3-b]pyridine-5-carboxylate (Preparation 54, 4.00 g), and the reaction mixture is heated to reflux for 18 h. The reaction mixture is allowed to cool to room temperature and is washed with water (200 mL) and brine (200 mL). The organic layer is concentrated in vacuo. The resulting solid is purified by column chromatography (CH₂Cl₂/methanol, 98/2; 97/3; 96/4) and then by recrystallization from EtOAc to yield 2.83 g of the title compound as a white solid. Physical characteristics. M.p. 111-112° C.; ¹H NMR (400 MHz, DMSO-d₆) δ 8.48-8.47, 7.80-7.76, 7.50-7.48, 7.27-7.24, 7.22, 5.37, 4.82-4.78, 4.21, 3.87-3.78, 2.83-2.79, 2.71-2.66, 2.31, 1.27; MS (ESI+) m/z 402 (M+H)⁺, HRMS (ESI) found 402.1467; [α]²⁵_{D}=+53° (c 0.95, methylene chloride). Anal. Found: C, 59.13; H, 5.89; N, 10.16; S, 7.98.

### Example 23

N-(4-Fluorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide.

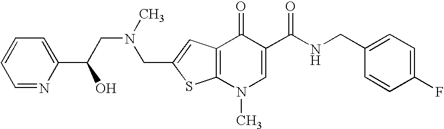

Ethyl 2-((((2R)-2-hydroxy-2-pyridin-2-ylethyl)(methyl)amino)methyl)-7-methyl-4-oxo-4,7-dihydrothieno[2,3-b]pyridine-5-carboxylate (Preparation 55, 0.400 g), 4-fluorobenzylamine (0.34 mL), and ethylene glycol (2 mL) are combined and heated to 130° C. for 18 h. The reaction mixture is allowed to cool to room temperature and is partitioned between water (20 mL) and CH₂Cl₂ (20 mL). The aqeuous layer is extracted with CH₂Cl₂ (3×20 mL). The combined organic layers are washed with brine (25 mL), dried (MgSO₄), filtered, and concentrated in vacuo. The residue is purified by column chromatography (CH₂Cl₂/methanol, 97/3) followed by recrystallization from EtOAc to yield 0.232 g of the title compound as a white solid. Physical characteristics. M.p. 139-1410C; ¹H NM (400 MHz, DMSO-d₆) δ 10.59, 8.70, 8.48-8.47, 7.80-7.76, 7.50-7.48, 7.38-7.35, 7.29, 7.27-7.25, 7.19-7.14, 5.38, 4.91, 4.83-4.79, 4.54, 3.92, 3.89-3.81, 2.84-2.80, 2.73-2.68, 2.32; MS (ESI+) m/z 481 (M+H)⁺; [α]²⁵_{D}=+41 (c 1.02, methylene chloride). Anal. Found: C, 62.12. H, 5.31; N, 11.52; S, 6.61.

### Example 24

N(4-Cyanobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide.

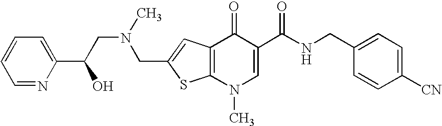

Ethyl 2-((((2R)-2-hydroxy-2-pyridin-2-ylethyl)(methyl)amino)methyl)-7-methyl-4-oxo-4,7-dihydrothieno[2,3-b]pyridine-5-carboxylate (Preparation 55, 0.400 g) is dissolved in methanol (10 mL). A solution of sodium methoxide in methanol (0.5 M, 2.0 mL) is added followed by 4-cyanobenzylamine (0.264 g, prepared as described in Biorg. Med. Chem. Lett. 2002, 12, 743-748). The reaction mixture is heated to 50° C. for 18 h and then allowed to cool to room temperature. The mixture is partitioned between water (25 mL) and CH₂Cl₂ (25 mL). The aqueous layer is extracted with CH₂Cl₂ (3×25 mL). The combined organic layers are dried (MgSO₄), filtered, and concentrated in vacuo. The resulting solid is purified by column chromatography (CH₂Cl₂/methanol, 98/2) followed by recrystallization from EtOAc to yield 0.122 g of the title compound as a white solid. Physical characteristics. M.p. 167-169° C. ¹H NM (400 MHz, DMSO-d₃) δ 10.69, 8.69, 8.48-8.47, 7.82-7.76, 7.51-7.49, 7.31, 7.27-7.24, 5.38, 4.83-4.78, 4.65, 3.90-3.82, 2.84-2.80, 2.73-2.68, 2.32; ¹³C NMR (100 MHz, DMSO-d₆) δ 171.8, 164.5, 163.2, 150.3, 148.1, 145.5, 145.1, 139.7, 136.3, 132.2, 130.4, 127.9, 122.0, 120.5, 119.8, 118.8, 114.1, 109.4, 72.0, 63.0, 56.2, 42.7, 42.3, 41.7; MS (ESI+) m/z 488 (M+H)⁺; [α]²⁵_{D}=+410 (c 0.81, methylene chloride). Anal. Found: C, 63.68; H, 5.12; N, 14.14; S, 6.43.

### Example 25

N(4-Bromobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide.

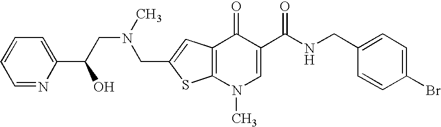

4-Bromobenzylamine hydrochloride (1.11 g) is dissolved in water (25 μL). The solution is adjusted to pH 12 with 2 NNaOH and extracted with CH₂Cl₂ (25 μL). The organic layer is concentrated in vacuo and the resulting free base is combined with ethyl 2-((((2R)-2-hydroxy-2-pyridin-2-ylethyl)(methyl)amino)methyl)-7-methyl-4-oxo-4,7-dihydrothieno[2,3-b]pyridine-5-carboxylate (Preparation 55, 0.200 g) and heated to 190° C. for 1 h. The reaction mixture is allowed to cool for several minutes and toluene (150 mL) is added. The mixture is concentrated in vacuo. The resulting residue is dissolved in CH₂Cl₂ (50 mL) and washed with water (50 mL). The organic layer is dried (MgSO₄), filtered, and concentrated in vacuo. The crude product is purified by column chromatography (CH₂Cl₂/methanol, 99/1; 98/2; 97/3) followed by recrystallization from EtOAc to yield 0.061 g of the title compound as a white solid. Physical characteristics. M.p. 151-153° C.; ¹H NMR (400 MHz, DMSO-d₆) δ 10.61, 8.69, 8.47-8.46, 7.79-7.76, 7.54-7.48, 7.29-7.24, 7.19-7.14, 5.37, 4.83-4.79, 4.53, 3.92, 3.89-3.81, 2.84-2.80, 2.72-2.67, 2.32; MS (ESI+) m/z 541 (M+H)⁺. Anal. Found: C, 55.11; H, 4.42; N, 10.24; Br, 14.60; S, 5.85.

We claim:

1. A compound of formula I, 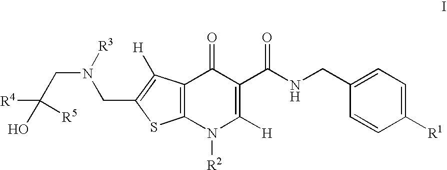
its enantiomeric, diasteromeric or tautomeric isomer, or a pharmaceutically acceptable salt thereof wherein,
R¹ is
(a) Cl,
(b) Br,
(c) F, or
(d) CN;
R² is
(a) C₁₋₄alkyl optionally substituted by one or more OH or C₁₋₄alkoxy, or
(b) (CH₂)ₘOCH₂CH₂OH;
R³ is C₁₋₂alkyl;
R⁴ is a six- (6) membered heteroaryl bonded via a carbon atom having 1, 2, or 3 nitrogen atoms, wherein R⁴ is optionally fused to a benzene ring, and optionally substituted with one or more R⁶;
R⁵ is
(a) H, or
(b) C₁₋₂alkyl optionally substituted by OH;
R⁶ is
(a) halo,
(b) OCF₃,
(c) cyano,
(d) nitro,
(e) CONR⁷R⁸,
(f) NR⁷R⁸,
(g) C₁₋₇alkyl, which is optionally partially unsaturated and is optionally substituted by one or more R⁹,
(h) O(CH₂CH₂O)ₙR¹⁰,
(i) OR¹⁰ or
(j) CO₂R¹⁰;
R⁷ and R⁸ are independently
(a) H,
(b) phenyl optionally substituted by halo, C₁₋₇alkyl, or C₁₋₇alkoxy,
(c) C₁₋₇alkyl which is optionally substituted by one or more OR¹⁰, phenyl, or halo substituents,
(d) C₃₋₈cycloalkyl,
(e) (C═O)R¹¹, or
(f) R⁷ and R⁸ together with the nitrogen to which they are attached form a het, wherein het is a five- (5), or six- (6) membered heterocyclic ring having 1, 2, or 3 heteroatoms selected from the group consisting of oxygen, sulfur, or nitrogen, wherein het is optionally substituted with C₁₋₄ alkyl;
R⁹ is
(a) oxo,
(b) phenyl optionally substituted by halo, C₁₋₇alkyl, or C₁₋₇alkoxy,
(c) OR¹⁰,
(d) O(CH₂CH₂)OR¹⁰,
(e) SR¹⁰,
(f) NR₇R₈,
(g) halo,
(h) CO₂R¹⁰,
(i) CONR¹⁰R¹⁰, or
(j) C₃₋₈cycloalkyl optionally substituted by OR¹⁰;
R¹⁰ is
(a) H,
(b) C₁₋₇alkyl,
(c) C₃₋₈cycloalkyl, or
(d) phenyl optionally substituted by halo, C₁₋₄alkyl, or C₁₋₇alkoxy;
R¹¹ is
(a) C₁₋₄alkyl,
(b) C₃₋₈cycloalkyl, or
(c) phenyl optionally substituted by halo, C₁₋₇alkyl, or C₁₋₇alkoxy;
n is 1, 2, 3, 4 or 5; and
m is 1 or 2.

2. A compound of claim 1 which is a compound of formula IA 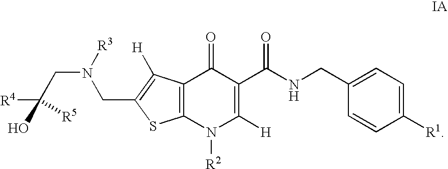
wherein, R¹, R², R³, R⁴, and R⁵ are as defined according to claim 1.

3. A compound of claim 1 wherein R¹ is chloro.

4. A compound of claim 1 wherein R² is C₁₋₃alkyl.

5. A compound of claim 1 wherein R² is methyl.

6. A compound of claim 1 wherein R² is C₁₋₃alkyl substituted with one or two hydroxy.

7. A compound of claim 1 wherein R² is C₁₋₄alkyl substituted by C₁₋₄alkoxy.

8. A compound of claim 1 wherein R³ is methyl.

9. A compound of claim 1 wherein R³ is ethyl.

10. A compound of claim 1 wherein R⁴ is a six- (6) membered heteroaryl bonded via a carbon atom having one (1) or two (2) nitrogen atoms.

11. A compound of claim 1 wherein R⁴ is a six- (6) membered heteroaryl bonded via a carbon atom having one (1) nitrogen atom.

12. A compound of claims 10 wherein R⁴ is substituted with R⁶.

13. A compound of claim 10 wherein R⁴ is pyridin-2-yl, pyridin-3-yl, pyridin-4-yl, pyrimidin-2-yl, pyridazin-3-yl, or pyrazin-2-yl.

14. A compound of claim 11 wherein R⁴ is pyridin-2-yl.

15. A compound of claim 13 wherein R⁴ is pyrimidin-2-yl.

16. A compound of claim 13 wherein R⁴ is pyrazin-2-yl.

17. A compound of claim 12 wherein R⁴ is 6-methylpyridin-2-yl.

18. A compound of claim 1 wherein R⁴ is a six- (6) membered heteroaryl bonded via a carbon atom having one (1) or two (2) nitrogen atoms and is fused to a benzene ring.

19. A compound of claim 18 wherein R⁴ is quinolin-2-yl.

20. A compound of claim 18 wherein R⁴ is substituted by R⁶.

21. A compound of claim 1 wherein R⁵ is hydrogen.

22. A compound of claim 12 or 20 wherein R⁶ is C₁₋₄alkyl, halo, C₁₋₄alkoxy, trifluoromethyl, or NR⁷R⁸.

23. A compound of claim 22 wherein R⁶ is methyl.

24. A compound of claim 22 wherein R⁶ is amino.

25. A pharmaceutical composition comprising a compound of claim 1 and a pharmaceutically acceptable carrier.

26. A method of treating infections by herpesviruses which comprises administering to a mammal in need thereof a compound of claim 1 or 2.

27. The method of claim 26 wherein said herpesviruses is herpes simplex virus types 1, herpes simplex virus types 2, varicella zoster virus, human cytomegalovirus, Epstein-Barr virus, human herpes virus 6, human herpes virus 7 or human herpes virus 8.

28. The method of claim 26 wherein said herpesviruses is human cytomegalovirus.

29. The method of claim 26 wherein said herpesviruses is varicella zoster virus or Epstein-Barr virus.

30. The method of claim 26 wherein said herpesviruses is herpes simplex virus types 1 or herpes simplex virus types 2.

31. The method of claim 26 wherein the compound of claim 1 is administered orally, parenterally or topically.

32. The method of claim 26 wherein the compound of claim 1 is in an amount of from about 0.1 to about 300 mg/kg of body weight.

33. The method of claim 26 wherein the compound of claim 1 is in an amount of from about 1 to about 30 mg/kg of body weight.

34. The method of claim 26 wherein said mammal is a human.

35. The method of claim 26 wherein said mammal is an animal.

36. A method of treating atherosclerosis and restenosis comprising administering to a mammal in need thereof a compound of claim 1 or 2.

37. A method for inhibiting a herpesviral DNA polymerase, comprising contacting the polymerase with an effective inhibitory amount of a compound of claim 1.

38. A compound of formula I, or a pharmaceutically acceptable salt thereof, for use in the manufacture of medicines for the treatment or prevention of a herpesviral infection in a maximal.

39. A compound of claim 1 which is
(1) rac-N-(4-chlorobenzyl)-2-(((2-hydroxy-2-pyridin-3-ylethyl)(methyl)amino)methyl)-7-methyl-4-oxo-4,7-dihydrothieno[2,3-b]pyridine-5-carboxamide,
(2) (+)-N-(4-chlorobenzyl)-2-(((2-hydroxy-2-pyridin-3-ylethyl)(methyl)amino)methyl)-7-methyl-4-oxo-4,7-dihydrothieno[2,3-b]pyridine-5-carboxamide,
(3) rac-N-(4-chlorobenzyl)-2-(((2-hydroxy-2-pyridin-4-ylethyl)(methyl)amino)methyl)-7-methyl-4-oxo-4,7-dihydrothieno[2,3-b]pyridine-5-carboxamide,
(4) rac-N-(4-chlorobenzyl)-2-(((2-hydroxy-2-pyridin-2-ylethyl)(methyl)amino)methyl)-7-methyl-4-oxo-4,7-dihydrothieno[2,3-b]pyridine-5-carboxamide,
(5) (+)-N-(4-chlorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide,
(6) rac-N-(4-chlorobenzyl)-2-(((2-hydroxy-2-(6-methylpyridin-2-yl)ethyl)(methyl)amino)methyl)-7-methyl-4-oxo-4,7-dihydrothieno[2,3-b]pyridine-5-carboxamide,
(7) rac-N-(4-chlorobenzyl)-2-(((2-hydroxy-2-quinolin-2-ylethyl)(methyl)amino)methyl)-7-methyl-4-oxo-4,7-dihydrothieno[2,3-b]pyridine-5-carboxamide,
(8) rac-N-(4-chlorobenzyl)-2-(((2-hydroxy-2-pyrimidin-2-ylethyl)(methyl)amino)methyl)-7-methyl-4-oxo-4,7-dihydrothieno[2,3-b]pyridine-5-carboxamide,
(9) N-(4-chlorobenzyl)-2-((((2R)-2-hydroxy-2-pyrimidin-2-ylethyl)(methyl)amino)methyl)-7-methyl-4-oxo-4,7-dihydrothieno[2,3-b]pyridine-5-carboxamide,
(10) rac-N-(4-chlorobenzyl)-2-(((2-hydroxy-2-pyrazin-2-ylethyl)(methyl)amino)methyl)-7-methyl-4-oxo-4,7-dihydrothieno[2,3-b]pyridine-5-carboxamide,
(11) N-(4-Chlorobenzyl)-2-((((2R)-2-hydroxy-2-pyrazin-2-ylethyl)(methyl)amino)methyl)-7-methyl-4-oxo-4,7-dihydrothieno[2,3-b]pyridine-5-carboxamide,
(12) N-(4-chlorobenzyl)-2-(((2-hydroxy-2-pyridazin-3-ylethyl)(methyl)amino)methyl)-7-methyl-4-oxo-4,7-dihydrothieno[2,3-b]pyridine-5-carboxamide,
(13) rac-N-(4-chlorobenzyl)-7-ethyl-2-(((2-hydroxy-2-pyrazin-2-ylethyl)(methyl)amino)methyl)-4-oxo-4,7-dihydrothieno[2,3-b]pyridine-5-carboxamide,
(14) rac-N-(4-chlorobenzyl)-7-ethyl-2-(((2-hydroxy-2-pyridin-2-ylethyl)(methyl)amino)methyl)-4-oxo-4,7-dihydrothieno[2,3-b]pyridine-5-carboxamide,
(15) rac-N-(4-chlorobenzyl)-7-propyl-2-(((2-hydroxy-2-pyridin-2-ylethyl)(methyl)amino)methyl)-4-oxo-4,7-dihydrothieno[2,3-b]pyridine-5-carboxamide,
(16) rac-N-(4-chlorobenzyl)-2-(((2-hydroxy-2-pyrazin-2-ylethyl) (methyl)amino)methyl)-4-oxo-7-propyl-4,7-dihydrothieno[2,3-b]pyridine-5-carboxamide,
(17) N-(4-chlorobenzyl)-7-(2,3-dihydroxypropyl)-2-((((2R)-2-hydroxy-2-pyridin-2-ylethyl)(methyl)amino)methyl)-4-oxo-4,7-dihydrothieno[2,3-b]pyridine-5-carboxamide,
(18) N-(4-chlorobenzyl)-7-(3-hydroxypropyl)-2-((((2R)-2-hydroxy-2-pyridin-2-ylethyl)(methyl)amino)methyl)-4-oxo-4,7-dihydrothieno[2,3-b]pyridine-5-carboxamide,
(19) rac-(4-chlorobenzyl)-7-(3-hydroxypropyl)-2-(((2-hydroxy-2-pyrimidin-2-ylethyl)(methyl)amino)methyl)-4-oxo-4,7-dihydrothieno[2,3-b]pyridine-5-carboxamide,
(20) N-(4-chlorobenzyl)-7-(2-hydroxyethyl)-2-((((2R)-2-hydroxy-2-pyridin-2-ylethyl)(methyl)amino)methyl)-4-oxo-4,7-dihydrothieno[2,3-b]pyridine-5-carboxamide,
(21) rac-N-(4-chlorobenzyl)-2-(((2-hydroxy-2-pyrazin-2-ylethyl)(methyl)amino)methyl)-7-(2-methoxyethyl)-4-oxo-4,7-dihydrothieno[2,3-b]pyridine-5-carboxamide,
(22) N-(4-Chlorobenzyl)-2-((((2R)-2-hydroxy-2-pyrazin-2-ylethyl)(methyl)amino)methyl)-4-oxo-7-(2-(2-(tetrahydro-2H-pyran-2-yloxy)ethoxy)ethyl)-4,7-dihydrothieno[2,3-b]pyridine-5-carboxamide,
(23) N-(4-fluorobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide,
(24) N-(4-cyanobenzyl)-2-((((2R)-2-hydroxy-2-pyridin-2-ylethyl)(ethyl)amino)methyl)-7-methyl-4-oxo-4,7-dihydrothieno[2,3-b]pyridine-5-carboxamide,
(25) N-(4-bromobenzyl)-2-((((2R)-2-hydroxy-2-pyridin-2-ylethyl)(methyl)amino)methyl)-7-methyl-4-oxo-4,7-dihydrothieno[2,3-b]pyridine-5-carboxamide, and a pharmaceutically acceptable salt thereof.

40. A compound of claim 39 which is rac-N-(4-chlorobenzyl)-2-(((2-hydroxy-2-pyridin-2-ylethyl)(methyl)amino)methyl)-7-methyl-4-oxo-4,7-dihydrothieno[2,3-b]-pyridine-5-carboxamide or a pharmaceutically acceptable salt thereof.

41. A compound of claim 39 which is (+)-N-(4-chlorobenzyl)-2-((((2R)-2-hydroxy-2-pyridin-2-ylethyl)(methyl)amino)methyl)-7-methyl-4-oxo-4,7-dihydrothieno[2,3-b]-pyridine-5-carboxamide or a pharmaceutically acceptable salt thereof.

42. A compound of claim 39 which is rac-N-(4-chlorobenzyl)-2-(((2-hydroxy-2-pyrimidin-2-ylethyl)(methyl)amino)methyl)-7-methyl-4-oxo-4,7-dihydrothieno[2,3-b]-pyridine-5-carboxamide, or a pharmaceutically acceptable salt thereof.

43. A compound of claim 39 which is N-(4-chlorobenzyl)-2-((((2R)-2-hydroxy-2-pyrimidin-2-ylethyl)(methyl)amino)methyl)-7-methyl-4-oxo-4,7-dihydrothieno[2,3-b]-pyridine-5-carboxamide, or a pharmaceutically acceptable salt thereof.

44. A method for preparing a compound of formula (I) according to claim 1 comprising:
(a) reacting an amine of a formula II, 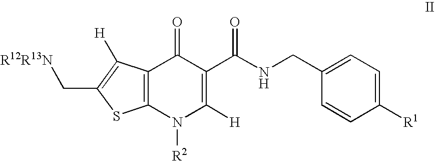
with ethylchloroformate to produce a compound of the formula III, 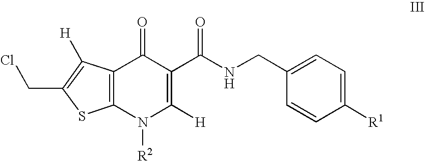
and (b) reacting a compound of formula III with an amino alcohol of the formula R⁴R⁵C(OH)CH₂NH(R³) in the presence of an inorganic or tertiary amine base; wherein,
R¹ is
(a) Cl,
(b) Br,
(c) F, or
(d) CN;
R² is
(a) C₁₋₄alkyl optionally substituted by one or more OH or C₁₋₄alkoxy, or
(b) (CH₂)ₘOCH₂CH₂OH;
R³ is C₁₋₂alkyl;
R⁴ is a six- (6) membered heteroaryl bonded via a carbon atom having 1, 2, or 3 nitrogen atoms, wherein R⁴ is optionally fused to a benzene ring, and optionally substituted with one or more R⁶;
R⁵ is
(a) H, or
(b) C₁₋₂alkyl optionally substituted by OH;
R⁶ is
(a) halo,
(b) OCF₃,
(c) cyano,
(d) nitro,
(e) CONR⁷R⁸,
(f) NR⁷R⁸,
(g) C₁₋₇alkyl, which is optionally partially unsaturated and is optionally substituted by one or more R⁹,
(h) O(CH₂CH₂O)ₙR¹⁰,
(i) OR¹⁰, or
(j) CO₂R¹⁰;
R⁷ and R⁸ are independently
(a) H,
(b) phenyl optionally substituted by halo, C₁₋₇alkyl, or C₁₋₇alkoxy,
(c) C₁₋₇alkyl which is optionally substituted by one or more OR¹⁰, phenyl, or halo substituents,
(d) C₃₋₈cycloalkyl,
(e) (C═O)R¹¹, or
(f) R¹ and R⁸ together with the nitrogen to which they are attached form a het, wherein het is a five- (5), or six- (6) membered heterocyclic ring having 1, 2, or 3 heteroatoms selected from the group consisting of oxygen, sulfur, or nitrogen, wherein het is optionally substituted with C₁₋₄ alkyl;
R⁹ is
(a) oxo,
(b) phenyl optionally substituted by halo, C₁₋₇alkyl, or C₁₋₇alkoxy,
(c) OR¹⁰,
(d) O(CH₂CH₂)OR¹⁰,
(e) SR¹⁰,
(f) NR₇R₈,
(g) halo,
(h) CO₂R¹⁰,
(i) CONR¹⁰R¹⁰, or
(j) C₃₋₈cycloalkyl optionally substituted by OR¹⁰;
R¹⁰ is
(a) H,
(b) C₁₋₇alkyl,
(c) C₃₋₈cycloalkyl, or
(d) phenyl optionally substituted by halo, C₁₋₄alkyl, or C₁₋₇alkoxy;
R¹¹ is
(a) C₁₋₇alkyl,
(b) C₃₋₈cycloalkyl, or
(c) phenyl optionally substituted by halo, C₁₋₇alkyl, or C₁₋₇alkoxy;
R¹² and R¹³ are independently C₁₋₇alkyl, or R¹² and R¹³ together with the nitrogen to which they are attached form morpholine, pyrrolidine, or piperidine;
n is 1, 2, 3, 4 or 5; and
m is 1 or 2.

45. A method according to claim 44 wherein R¹² and R¹³ together with the nitrogen to which they are attached form morpholine.

46. A method according to claim 44 wherein R¹² and R¹³ are independently methyl.

47. A method according to claim 44 wherein R¹ is chloro, R² and R³ are independently methyl, R⁴ is pyridin-2-yl, and R⁵ is hydrogen.

48. A method according to claim 44 wherein R² is chloro, R² and R³ are methyl, R⁴ is pyrimidin-2-yl, and R¹ is hydrogen.

49. N-(4-Chlorobenzyl)-2-(chloromethyl)-7-methyl-4-oxo-4,7-dihydrothieno[2,3-b]pyridine-5-carboxamide.
